(19) United States
(12) Patent Application Publication
(10) Pub. No.: US 2025/0326706 A1
(43) Pub. Date: Oct. 23, 2025

(54) METHODS AND COMPOSITIONS USEFUL FOR OXIDATION OF BIOMASS TO CARBOXYLIC ACIDS USING POLYOXOMETALATE IONIC LIQUIDS

(71) Applicants: THE REGENTS OF THE UNIVERSITY OF CALIFORNIA, Oakland, CA (US); NATIONAL TECHNOLOGY & ENGINEERING SOLUTIONS OF SANDIA, LLC, Albuquerque, NM (US)

(72) Inventors: Hemant CHOUDHARY, Emeryville, CA (US); Mood MOHAN, Emeryville, CA (US); Blake A. SIMMONS, San Francisco, CA (US); John M. GLADDEN, Alameda, CA (US); Seema SINGH, Hayward, CA (US)

(21) Appl. No.: 19/180,503

(22) Filed: Apr. 16, 2025

Related U.S. Application Data
(60) Provisional application No. 63/635,312, filed on Apr. 17, 2024.

(51) Int. Cl. C07C 51/295

(52) U.S. Cl. CPC C07C 51/295

(57) ABSTRACT

The present invention provides for a method to deconstruct a biomass: the method comprising: introducing a solvent comprising a polyoxometalate (POM) ionic liquid (POM-IL) to a biomass to produce a mixture, such that the POM-IL oxidizes the biomass to produce a carboxylic acid.

### CROSS-REFERENCE TO RELATED APPLICATIONS

This application claims priority to U.S. Provisional Patent Application Ser. No. 63/635,312, filed Apr. 17, 2024, which is incorporated by reference in its entirety.

### STATEMENT OF GOVERNMENTAL SUPPORT

The invention was made with government support under Contract No. DE-AC02-05CH11231 awarded by the U.S. Department of Energy. The government has certain rights in the invention.

### FIELD OF THE INVENTION

The present invention is in the field of biomass pretreatment.

### BACKGROUND OF THE INVENTION

The realization of sustainable and competitive biorefineries is indispensable to an effective utilization of lignin, a fractious heterogeneous aromatic polymer.^{[1,2]} Exploiting biochemical pathways is attractive for industrial implementation due to potential lower operational cost and environmental impact.^{[3]} Nevertheless, the limited biological pathways for polymeric lignin valorization restricts industrial application.^{[4]} Therefore, an integrated approach involving chemical breakdown of polymeric lignin to enable the bioconversion of depolymerized fractions could be a viable solution.

In this regard, the extensively explored reductive catalytic fractionation has emerged as dominant and promising for producing phenolic chemicals, but due to their toxicity to organisms they are not suitable for bioconversion.^{[5 9]} Other depolymerization approaches, for example oxidative catalytic fractionation or base-catalyzed depolymerization, can produce aromatic acids. Rodriguez et. al showcased a successful consumption of aromatic acids, but ultimately overall only 15% lignin conversion and consumption was achieved.^{[10-13]}

The bioconversion/metabolism of aliphatic acids is well known and they can be integrated into the Krebs cycle. For producing these bioavailable aliphatic acids, the ring opening reaction for dearomatization is promising.

The ring opening reaction has been explored on lignin model compounds and the use of H₂O₂ in combination with a metal catalyst has resulted in the production of organic acids.^{[14-20]} Although some of these studies reported the depolymerization to organic acids, only Martinez et al. explored their bioconversion.

A promising class of catalysts are polyoxometalates (POM), which have been recognized for their reversable redox capabilities and tunable Brønsted/Lewis acidity.^{[21,22]} Especially molybdenum and tungsten polyoxoanions have been employed in a wide range of applications.^{[22-24]} Recently the intermolecular interactions between tungsten-POM-IL ([C₄C₁im]₃ [PW₁₂O₄₀]) and Guaiacylglycerol-β-guaiacyl ether (GGE) were simulated to understand the lignin dissolution mechanism.^{[25]} Gregorio et al. reported a novel Polyoxometalate-Ionic Liquid catalyst ([PV₂Mo₁₀O₄₀]⁵⁻ 5 [HC₄Im]⁺) for depolymerizing lignin to vanillin and syringaldehyde with a maximum yield of 201 ppm, but noted that further research is necessary to exploit the full potential from this new class of catalysts.^{[26]}

### SUMMARY OF THE INVENTION

The present invention provides for a method to deconstruct a biomass: the method comprising: introducing a solvent comprising a polyoxometalate (POM) ionic liquid (POM-IL) to a biomass to produce a mixture, such that the POM-IL oxidizes the biomass to produce a carboxylic acid. In some embodiments, the solvent oxidizes at least a part of the biomass.

In some embodiments, the polyoxometalate (POM) is a polyatomic anion that consists of three or more transition metal oxyanions linked together by shared oxygen atoms to form closed 3-dimensional frameworks. In some embodiments, the metal atoms are group 6 (such as Mo or W), group 5 (such as V, Nb, or Ta), or group 7 (such as Tc or Re) transition metals in their high oxidation states. In some embodiments, the POM may contain a homopolymetalate or heteropolymetalate oxides with or without main group element, such as N, P, or Si. In some embodiments, the metal atoms are cations within the POM. In some embodiments, the POM is PV₂Mo₁₀O₄₀ or PW₁₂O₄₀.

In some embodiments, the method comprises or consists only one step. In some embodiments, the biomass is a lignocellulosic biomass. In some embodiments, a lignin of the biomass is oxidized into aromatic and/or organic acid(s). The aromatic acids and organic acids are carboxylic acids.

In some embodiments, the carboxylic acid is an aliphatic carboxylic acid or aromatic carboxylic acid, or a mixture thereof. In some embodiments, the carboxylic acid has 1, 2, 3, 4, 5, 6, 7, 8, 9, or 10 carbon atoms, or a mixture thereof. In some embodiments, the aliphatic carboxylic acid is a monocarboxylic acid, including but not limited to formic, acetic, malic, and propionic acids, or a mixture thereof. In some embodiments, the aliphatic carboxylic acid is a polycarboxylic acid. In some embodiments, the polycarboxylic acid is a dicarboxylic acid, including but not limited to oxalic, malonic, succinic, malic, and tartaric acids, or a mixture thereof. In some embodiments, the aromatic acid is a hydroxybenzoic acid, vanillic acid, p-coumaric acid, ferulic acid, syringic acid, or a mixture thereof.

In some embodiments, the introducing step comprises heating the mixture for at least 1 minute. In some embodiments, the introducing step comprises heating the mixture to a temperature of about 80, 100, 120, 140, 160, 180, or 200° C., or within a range of any two preceding two values. In some embodiments, the introducing step comprises heating the mixture for at least 5, 10, 15, 20, 25, 30, 35, 40, 45, 50, 55, 60, 90, or 120 minute, or within a range of any two preceding two values. In some embodiments, the introducing step comprises adding an oxidizing agent (such as hydrogen peroxide (H₂O₂)) to the mixture to about 1, 2, 3, 4, 5, 6, 7, 8, 9, or 10 wt %, or within a range of any two preceding two values.

In some embodiments, the method results in the mixture comprising a carboxylic acid, or mixture thereof, and unreacted or not oxidized biomass. In some embodiments, the lignocellulosic biomass comprises untreated and/or raw agricultural and forest residue, or residues, such as lignin residues, obtained from a biorefinery or paper and pulp. In some embodiments, the method deconstructs untreated and/or raw biomass to a carboxylic acid in aqueous and glucan rich solid residue In some embodiments, the method further comprises: separating the carboxylic acid from the mixture. In some embodiments, the carboxylic acid is biocompatible for further conversion.

This invention disclosure describes a unique approach to deconstruct lignocellulosic biomass employing polyoxometalate ionic liquids (POM-ILs) in subcritical water under mild conditions. This approach provided a facile pathway to afford fermentable sugars and organic acids as a function of the reaction coordinates.

Our industrialized society intends to smoothly transition from fossil fuels to renewable and human-inedible lignocellulosic biomass (composed of cellulose, hemicellulose, and lignin) to sustain the energy and chemical supply for the future. Nevertheless, the structural complexity of biomass demands cutting-edge technologies to enable facile conversion of lignocellulose in to value-added products. Current state-of-art employs multi-step conversion including a) separation of biopolymers, b) saccharification of holocelluloses, c) sugar fermentation, and d) lignin conversion. In this regard, a one-step method that can fractionate and convert these biopolymers into value-added chemicals under milder conditions is highly desired.

Polyoxometalates (POMs, transition metal(s) oxide anionic clusters composed of do metal cations) have been recognized as commercially viable option to oxidize lignin into aromatics and organic acids. POMs being an anion can be coupled with asymmetric cations to afford low melting salts commonly known as ionic liquids (ILs). On the other hand, subcritical water (pressurized hot water to maintain liquid state) offers characteristics unique to organic solvents in terms of extraction, hydrolysis, and the like, along with being non-toxic, non-flammable, and non-explosive. To develop a one-step deconstruction method, utilize all components, catalytic amount of POM-IL(s) is employed in subcritical water for oxidation of the lignocellulosic biomass.

In some embodiments, the method comprises oxidation of at least about 10%, 20%, 30%, 40%, 50%, 60%, 70%, 80%, 90%, or 100% of the biomass into organic acids over POM-ILs in subcritical water under mild conditions. In some embodiments, the oxidation of biomass, such as sorghum (grass) and/or pine (softwood), is subjected to about 140° C. for about 1 h over about 1-4 wt % POM-ILs (such as cholinium phosphotungstate or cholinium phosphomolybdate). In some embodiments, the method produces at least or up to about 10, 20, 30, 40, 50, 60, 70, 80, 90, 100, 150, 200, 250, 300, 350, 400, 450, 500, 600, or 700 g/L carboxylic acid, or any range within any two preceding values. In some embodiments, the method produces at least or up to about 50, 100, 150, 200, 250, 300, 350, 400, 450, or 500 g/L formic acid, or any range within any two preceding values. In some embodiments, the method produces at least or up to about 10, 20, 30, 40, 50, 60, 70, 80, 90, or 100 g/L acetic acid, or any range within any two preceding values. In some embodiments, the method produces up to about 345 g/L formic acid and/or about 81 g/L acetic acid. In some embodiments, the POM-IL is about 2-3 wt % POM-IL, or 1.5-2.5 wt % POM-IL. In some embodiments, only a small amount of solid (such as comprising of lower fractions of holocellulose and lignin) is recovered after the oxidation reaction.

We have demonstrated the facile oxidation of whole biomass into organic acids over POM-ILs in subcritical water under mild conditions. The oxidation of sorghum (grass) and pine (softwood) at 140° C. for 1 h over 2 wt % POM-ILs (cholinium phosphotungstate or cholinium phosphomolybdate) rendered up to 345 g/L and 81 g/L formic and acetic acids. Only a small amount of solid (comprising of lower fractions of holocellulose and lignin) was recovered after the oxidation reaction implying high conversion of biomass. In the absence of oxidizing agents, up to 90 g/L of reducing sugars were recorded. We also attempted the oxidation of organosolv lignin to realize similar oxidation product profile.

The current invention offers several economic advantages; however, the key differences form the current state-of-art are enlisted here: (1) One-step low temperature conversion of whole biomass into value-added products. (2) Reduced number of steps for the biomass processing. (3) Possible lower cost of the whole process. (4) Possible recycling of POM-ILs. (5) Control over aromatic and hydrocarbon product profile as a function of reaction coordinates.

In some embodiments, the method further comprises separating a solubilized biomass from the POM-IL; optionally introducing an enzyme and/or a microbe to the solubilized biomass such that the enzyme and/or microbe produces a sugar from the solubilized biomass; and, optionally separating the sugar from the solubilized biomass.

In some embodiments, the method further comprises forming the POM-IL prior to the introducing step, and optionally forming the solvent by providing the POM-IL.

The present invention provides for a composition comprising: (a) a solvent comprising a POM-IL, and (b) a biomass.

In some embodiments, the composition comprises a mixture comprising a polyoxometalate (POM) ionic liquid (POM-IL) and a biomass. In some embodiments, the composition further comprises a carboxylic acid, wherein the carboxylic acid is produced from the POM-IL oxidizing the biomass.

In some embodiments, the present invention is used to convert waste biomass (from agricultural residues, wood/paper/pulping, grasses) into biofuels and/or bioproducts. In some embodiments, the process helps achieve a high concentration of fermentable sugars while leaving the residual lignin for valuable chemicals.

In some embodiments, the method further comprises ensiling a biomass, prior to the introducing step, to produce an ensiled biomass comprising one or more organic acids, wherein the ensile biomass is the biomass of the introducing step. In some embodiments, the ensiled biomass comprises equal to or more than about 10%, 20%, 30%, or 40% by weight of the one or more organic acids. In some embodiments, the one or more organic acids comprises an alkanoic acid. In some embodiments, the alkanoic acid is lactic acid, acetic acid, butyric acid, or propionic acid, or a mixture thereof. In some embodiments, the ensiling step produces one or more toxic compounds in the ensiled biomass, and the microbe is resistant to the one or more toxic compounds. In some embodiments, the one or more toxic compound is an organic acid, such as a straight chained or branched alkanoic acid (such as acetic acid, lactic acid, or formic acid), or an aromatic organic acid (such as benzoic acid, vanillic acid, or the like). In some embodiments, the organic acid has between about 2 to 10 carbon atoms.

In some embodiments, the method further comprises one or more steps taught in U.S. Provisional Patent Application Ser. No. 63/016,877, filed Apr. 28, 2020, and U.S. patent application Ser. No. 17/242,256, filed Apr. 27, 2021 (both are hereby incorporated by reference in their entireties.

In some embodiments, the method further comprises: introducing an enzyme and/or a microbe to the solubilized biomass mixture such that the enzyme and/or microbe produces a sugar from the solubilized biomass mixture. In some embodiments, the method further comprises: separating the sugar from the solubilized biomass mixture.

The present invention provides for compositions and methods described herein. In some embodiments, the compositions and methods further comprise steps, features, and/or elements described in U.S. patent application Ser. No. 16/737,724, hereby incorporated by reference in its entirety. The present invention provides for compositions and methods described herein.

In some embodiments, the POM-IL, IL and/or DES are bio-compatible.

### BRIEF DESCRIPTION OF THE DRAWINGS

The foregoing aspects and others will be readily appreciated by the skilled artisan from the following description of illustrative embodiments when read in conjunction with the accompanying drawings.

FIG. 1. Oxidative catalysis of OL with POM-IL.

FIG. 2. Fermentation with oxidized stream from OL as carbonsource in YNB media; purple=YNB+oxidized stream, black=negative control only YNB.

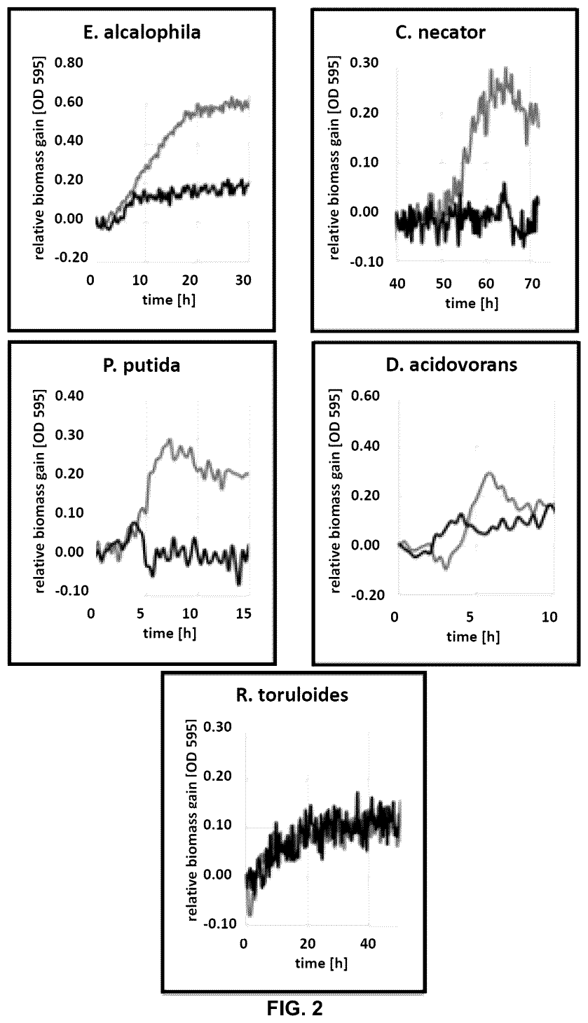

FIG. 3. Biomass gain after respective time points for bioconversion of oxidized stream from sorghum lignin.

FIG. 4. IR spectrum of synthesized catalyst.

FIG. 5. GPC of sorghum lignin.

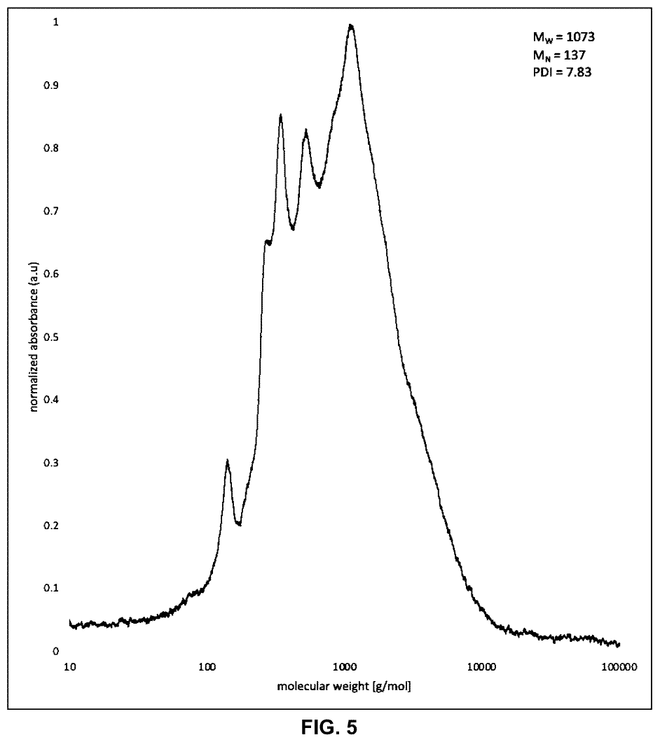

FIG. 6. GPC plot of control reactions.

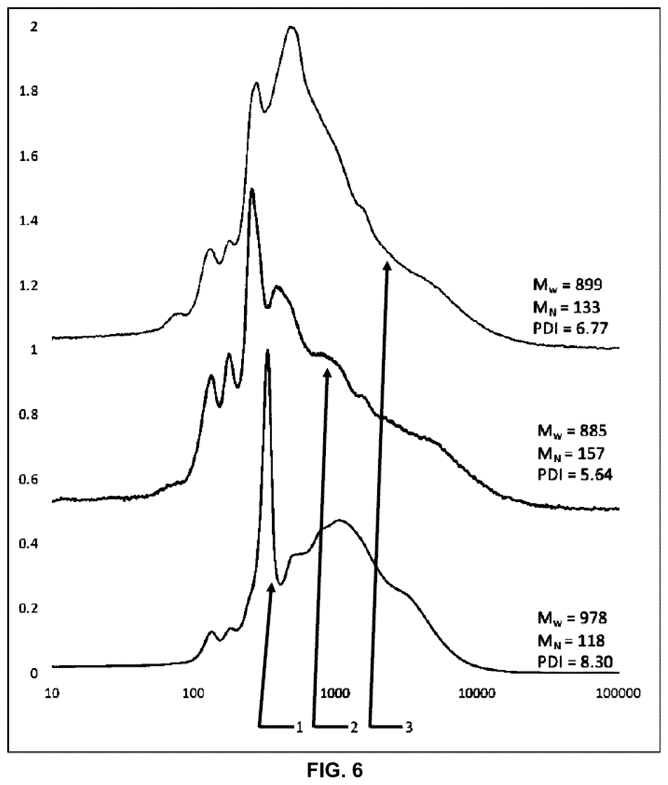

FIG. 7. GPC plot of sorghum lignin reactions entry 7-10.

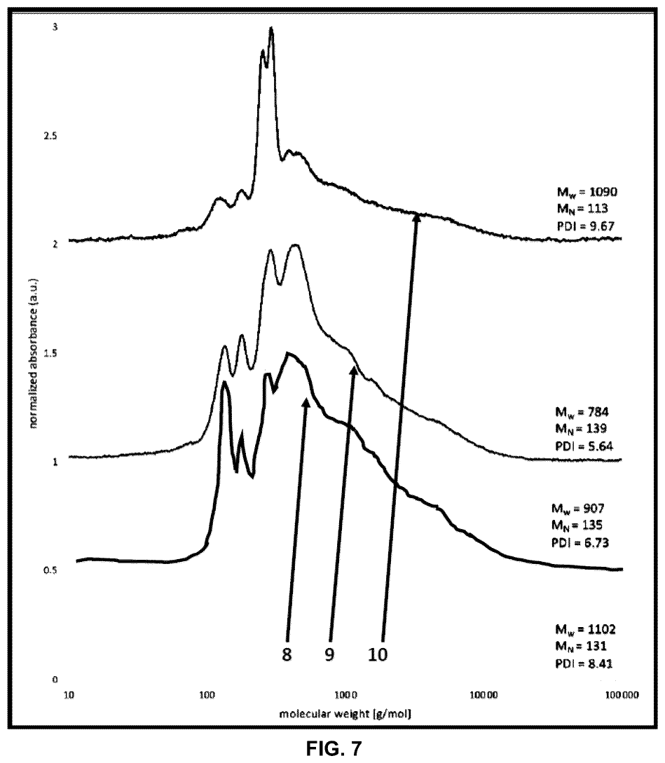

FIG. 8. GPC plots of raw biomass and their respective residual solids.

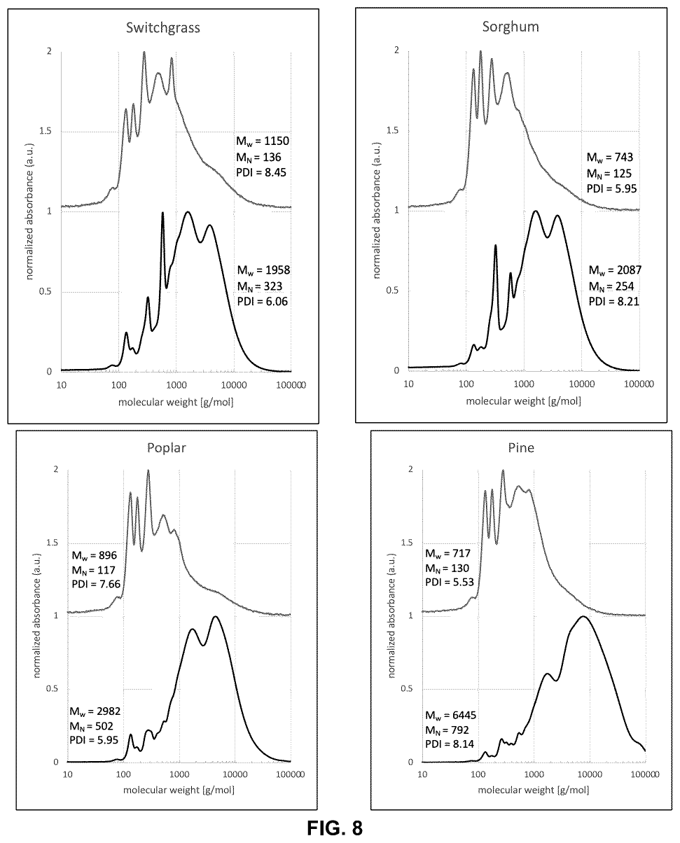

### DETAILED DESCRIPTION OF THE INVENTION

Before the invention is described in detail, it is to be understood that, unless otherwise indicated, this invention is not limited to particular sequences, expression vectors, enzymes, host microorganisms, or processes, as such may vary. It is also to be understood that the terminology used herein is for purposes of describing particular embodiments only, and is not intended to be limiting.

In this specification and in the claims that follow, reference will be made to a number of terms that shall be defined to have the following meanings:

The terms “optional” or “optionally” as used herein mean that the subsequently described feature or structure may or may not be present, or that the subsequently described event or circumstance may or may not occur, and that the description includes instances where a particular feature or structure is present and instances where the feature or structure is absent, or instances where the event or circumstance occurs and instances where it does not.

The term “about” when applied to a value, describes a value that includes up to 10% more than the value described, and up to 10% less than the value described.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

In some embodiments, the introducing step takes place in a vessel and homogenized. In some embodiments, the loading is solid loading and controlled at about 5%, 10%, 15%, 20%, 25%, 30%, 35%, or 40%, or a range within any two preceding values. In some embodiments, the biomass and the solvent are heated, such as to 100° C., 110° C., 120° C., 130° C., 140° C., 150° C., 160° C., 170° C., 180° C., 190° C., 200° C., 200° C., 212° C., or a range within any two preceding values, for a period of time, such as about 1 h, 2 h, 3 h, 4 h, or 5 h, or a range within any two preceding values. In some embodiments, after pretreatment, the mixture is cooled, such as for a period of about at least 30 mins, such as at room temperature, or about 25° C., and/or then washed at least about 1×, 2×, 3×, 4×, or 5× with water, such as deionized water. In some embodiments, the resulting solid is recovered, such as separating the solid portion with the liquid portion.

In some embodiments, the biomass is a lignocellulosic biomass. In some embodiments, the vessel is made of a material that is inert, such as stainless steel or glass, that does not react or interfere with the reactions in the pretreatment mixture.

In some embodiments, the method further comprises heating the mixture, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel, bioproduct and/or chemical compound. In some embodiments, there is a plurality of microbes. In some embodiments, the method produces a sugar and a lignin from the biomass. The sugar is used for growth by the microbe.

In some embodiments, the solubilizing is full, near full (such as at least about 70, 80, or 90%), or partial (such as at least about 10, 20, 30, 40, 50, or 60%). In some embodiments, the mixture is a slurry.

### Ionic Liquid

Ionic liquids (ILs) are salts that are liquids rather than crystals at room temperatures. It will be readily apparent to those of skill that numerous ILs can be used in the present invention. In some embodiments of the invention, the IL is suitable for pretreatment of the biomass and for the hydrolysis of cellulose by thermostable cellulase. Suitable ILs are taught in ChemFiles (2006) 6 (9) (which are commercially available from Sigma-Aldrich, Milwaukee, Wis.). Such suitable ILs include, but are not limited to, 1-alkyl-3-alkylimidazolium alkanate, 1-alkyl-3-alkylimidazolium alkylsulfate, 1-alkyl-3-alkylimidazolium methylsulfonate, 1-alkyl-3-alkylimidazolium hydrogensulfate, 1-alkyl-3-alkylimidazolium thiocyanate, and 1-alkyl-3-alkylimidazolium halide, wherein an “alkyl” is an alkyl group comprising from 1 to 10 carbon atoms, and an “alkanate” is an alkanate comprising from 1 to 10 carbon atoms. In some embodiments, the “alkyl” is an alkyl group comprising from 1 to 4 carbon atoms. In some embodiments, the “alkyl” is a methyl group, ethyl group or butyl group. In some embodiments, the “alkanate” is an alkanate comprising from 1 to 4 carbon atoms. In some embodiments, the “alkanate” is an acetate. In some embodiments, the halide is chloride.

In some embodiments, the IL includes, but is not limited to, 1-ethyl-3-methylimidazolium acetate (EMIN Acetate), 1-ethyl-3-methylimidazolium chloride (EMIN Cl), 1-ethyl-3-methylimidazolium hydrogensulfate (EMIM HOSO₃), 1-ethyl-3-methylimidazolium methylsulfate (EMIM MeOSO₃), 1-ethyl-3-methylimidazolium ethylsulfate (EMIM EtOSO₃), 1-ethyl-3-methylimidazolium methanesulfonate (EMIM MeSO₃), 1-ethyl-3-methylimidazolium tetrachloroaluminate (EMIM AlCl₄), 1-ethyl-3-methylimidazolium thiocyanate (EMIM SCN), 1-butyl-3-methylimidazolium acetate (BMIM Acetate), 1-butyl-3-methylimidazolium chloride (BMIM Cl), 1-butyl-3-methylimidazolium hydrogensulfate (BMIM HOSO₃), 1-butyl-3-methylimidazolium methanesulfonate (BMIM MeSO₃), 1-butyl-3-methylimidazolium methylsulfate (BMIM MeOSO₃), 1-butyl-3-methylimidazolium tetrachloroaluminate (BMIM AlCl₄), 1-butyl-3-methylimidazolium thiocyanate (BMIM SCN), 1-ethyl-2,3-dimethylimidazolium ethylsulfate (EDIM EtOSO₃), Tris(2-hydroxyethyl)methylammonium methylsulfate (MTEOA MeOSO₃), 1-methylimidazolium chloride (MIM Cl), 1-methylimidazolium hydrogensulfate (MIM HOSO₃), 1,2,4-trimethylpyrazolium methylsulfate, tributylmethylammonium methylsulfate, choline acetate, choline salicylate, and the like.

In some embodiments, the ionic liquid is a chloride ionic liquid. In other embodiments, the ionic liquid is an imidazolium salt. In still other embodiments, the ionic liquid is a 1-alkyl-3-imidazolium chloride, such as 1-ethyl-3-methylimidazolium chloride or 1-butyl-3-methylimidazolium chloride.

In some embodiments, the ionic liquids used in the invention are pyridinium salts, pyridazinium salts, pyrimidium salts, pyrazinium salts, imidazolium salts, pyrazolium salts, oxazolium salts, 1,2,3-triazolium salts, 1,2,4-triazolium salts, thiazolium salts, isoquinolium salts, quinolinium salts isoquinolinium salts, piperidinium salts and pyrrolidinium salts. Exemplary anions of the ionic liquid include, but are not limited to halogens (e.g., chloride, fluoride, bromide and iodide), pseudohalogens (e.g., azide and isocyanate), alkyl carboxylate, sulfonate, acetate and alkyl phosphate.

Additional ILs suitable for use in the present invention are described in U.S. Pat. Nos. 6,177,575; 9,765,044; and, 10,155,735; U.S. Patent Application Publication Nos. 2004/0097755 and 2010/0196967; and, PCT International Patent Application Nos. PCT/US2015/058472, PCT/US2016/063694, PCT/US2017/067737, and PCT/US2017/036438 (all of which are incorporated in their entireties by reference). It will be appreciated by those of skill in the art that others ILs that will be useful in the process of the present invention are currently being developed or will be developed in the future, and the present invention contemplates their future use. The ionic liquid can comprise one or a mixture of the compounds.

In some embodiments, the IL is a protic ionic liquid (PIL). Suitable protic ionic liquids (PILs) include fused salts with a melting point less than 100° C. with salts that have higher melting points referred to as molten salts. Suitable PPILs are disclosed in Greaves et al. “Protic Ionic Liquids: Properties and Applications” Chem. Rev. 108 (1): 206-237 (2008). PILs can be prepared by the neutralization reaction of certain Brønsted acids and Brønsted bases (generally from primary, secondary or tertiary amines, which are alkaline) and the fundamental feature of these kinds of ILs is that their cations have at least one available proton to form hydrogen bond with anions. In some embodiments, the protic ionic liquids (PILs) are formed from the combination of organic ammonium-based cations and organic carboxylic acid-based anions. PILs are acid-base conjugate ILs that can be synthesized via the direct addition of their acid and base precursors. In some embodiments, the PIL is a hydroxyalkylammonium carboxylate. In some embodiments, the hydroxyalkylammonium comprises a straight or branched C1, C2, C3, C4, C5, C6, C7, C8, C9, or C10 chain. In some embodiments, the carboxylate comprises a straight or branched C1, C2, C3, C4, C5, C6, C7, C8, C9, or C10 chain. In some embodiments, the carboxylate is substituted with one or more hydroxyl groups. In some embodiments, the PIL is a hydroxyethylammonium acetate.

In some embodiments, the protic ionic liquid (PIL) is disclosed by U.S. Patent Application Publication No. 2004/0097755, hereby incorporated by reference.

Suitable salts for the method include combinations of organic ammonium-based cations (such as ammonium, hydroxyalkylammonium, or dimethylalkylammonium) with organic carboxylic acid-based anions (such as acetic acid derivatives (C1-C8), lactic acid, glycolic acid, and DESs such as ammonium acetate/lactic acid).

Suitable IL, such as distillable IL, are disclosed in Chen et al. “Distillable Ionic Liquids: reversible Amide O Alkylation”, Angewandte Comm. 52:13392-13396 (2013), King et al. “Distillable Acid-Base Conjugate Ionic Liquids for Cellulose Dissolution and Processing”, Angewandte Comm. 50:6301-6305 (2011), and Vijayaraghavan et al. “CO₂-based Alkyl Carbamate Ionic Liquids as Distillable Extraction Solvents”, ACS Sustainable Chem. Engin. 2:31724-1728 (2014), all of which are hereby incorporated by reference.

Suitable PIL, such as distillable PIL, are disclosed in Idris et al. “Distillable Protic Ionic Liquids for Keratin Dissolution and Recovery”, ACS Sustainable Chem. Engin. 2:1888-1894 (2014) and Sun et al. “One-pot integrated biofuel production using low-cost biocompatible protic ionic liquids”, Green Chem. 19 (13): 3152-3163 (2017), all of which are hereby incorporated by reference.

In some embodiments, the PILs are formed with the combination of organic ammonium-based cations and organic carboxylic acid-based anions. PILs are acid-base conjugate ILs that can be synthesized via the direct addition of their acid and base precursors. Additionally, when sufficient energy is employed, they can dissociate back into their neutral acid and base precursors, while the PILs are re-formed upon cooling. This presents a suitable way to recover and recycle the ILs after their application. In some embodiments, the PIL (such as hydroxyethylammonium acetate—[Eth][OAc]) is an effective solvent for biomass pretreatment and is also relatively cheap due to its ease of synthesis (Sun et al., Green Chem. 19 (13): 3152-3163 (2017)).

### Deep Eutectic Solvent (DES)

DESs are systems formed from a eutectic mixture of Lewis or Brønsted acids and bases which can contain a variety of anionic and/or cationic species. DESs can form a eutectic point in a two-component phase system. DESs are formed by complexation of quaternary ammonium salts (such as, choline chloride) with hydrogen bond donors (HBD) such as amines, amides, alcohols, or carboxylic acids. The interaction of the HBD with the quaternary salt reduces the anion-cation electrostatic force, thus decreasing the melting point of the mixture. DESs share many features of conventional ionic liquid (IL), and promising applications would be in biomass processing, electrochemistry, and the like. In some embodiments, the DES is any combination of Lewis or Brønsted acid and base. In some embodiments, the Lewis or Brønsted acid and base combination used is distillable.

In some embodiments, DES is prepared using an alcohol (such as glycerol or ethylene glycol), amines (such as urea), and an acid (such as oxalic acid or lactic acid). The present invention can use renewable DESs with lignin-derived phenols as HBDs. Both phenolic monomers and phenol mixture readily form DES upon heating at 100° C. with specific molar ratio with choline chloride. This class of DES does not require a multistep synthesis. The DES is synthesized from lignin which is a renewable source.

Both monomeric phenols and phenol mixture can be used to prepare DES. DES is capable of dissolving biomass or lignin, and can be utilized in biomass pretreatment and other applications. Using DES produced from biomass could lower the cost of biomass processing and enable greener routes for a variety of industrially relevant processes.

The DES, or mixture thereof, is bio-compatible: meaning the DES, or mixture thereof, does not reduce or does not significantly reduce the enzymatic activity of the enzyme, and/or is not toxic, and/or does not reduce or significantly reduce, the growth of the microbe. A “significant” reduction is a reduction to 70, 80, 90, or 95% or less of the enzyme's enzymatic activity and/or the microbe's growth (or doubling time), if the DES, or mixture thereof, was not present.

In some embodiments, the DES, or mixture thereof, comprises a quaternary ammonium salt and/or glycerol. In some embodiments, the DES, or mixture thereof, comprises a quaternary ammonium salt and/or glycerol. In some embodiments, the quaternary ammonium salt and/or glycerol have a molar ratio of about 1:1 to about 1:3. In some embodiments, the quaternary ammonium salt and/or glycerol have a molar ratio of about 1:1.5 to about 1:2.5. In some embodiments, the quaternary ammonium salt and/or glycerol have a molar ratio of about 1:1.8 or 1:1.9 to about 1:2.1 or 1:2.2. In some embodiments, the quaternary ammonium salt and/or glycerol have a molar ratio of about 1:2. In some embodiments, the quaternary ammonium salt is a choline halide, such choline chloride.

In some embodiments, the DES is distillable if the DES can be recovered at least equal to or more than about 50%, 55%, 60%, 65%, 70%, 75%, 80%, or 85% yield by distilling over vacuum at a temperature at about 100° C., 110° C., 120° C., 130° C., 140° C., 150° C., or 160° C., or any temperature between any two of the preceding temperatures.

In some embodiments, the DES can be one taught in WO 2018/204424 (Seema Singh et al.), which is hereby incorporated in its entirety by reference.

In some embodiments, the method further comprises heating the one-pot composition, optionally also comprising the enzyme and/or microbe, to a temperature that is equal to, about, or near the optimum temperature for the enzymatic activity of the enzyme and/or growth of the microbe. In some embodiments, the enzyme is a genetically modified host cell capable of converting the cellulose in the biomass into a sugar. In some embodiments, there is a plurality of enzymes. In some embodiments, the microbe is a genetically modified host cell capable of converting a sugar produced from the biomass into a biofuel and/or chemical compound. In some embodiments, there is a plurality of microbes. In some embodiments, the introducing step(s) produce a sugar and a lignin from the biomass. The lignin can further be processed to produce a DES. The sugar is used for growth by the microbe.

In some embodiments, the solubilizing is full, near full (such as at least about 70, 80, or 90%), or partial (such as at least about 10, 20, 30, 40, 50, or 60%). In some embodiments, the one-pot composition is a slurry. When the steps described herein are continuous, the one-pot composition is in a steady state.

In some embodiments, the introducing step comprises heating the mixture comprises increasing the temperature of the solution to a value within a range of about 75° C. to about 125° C. In some embodiments, the heating step comprises increasing the temperature of the solution to a value within a range of about 80° C. to about 120° C. In some embodiments, the heating step comprises increasing the temperature of the solution to a value within a range of about 90° C. to about 110° C. In some embodiments, the heating step comprises increasing the temperature of the solution to about 100° C.

### Enzyme

In some embodiments, the enzyme is a cellulase. In some embodiments, the enzyme is thermophilic or hyperthermophilic. In some embodiments, the enzyme is any enzyme taught in U.S. Pat. Nos. 9,322,042; 9,376,728; 9,624,482; 9,725,749; 9,803,182; and 9,862,982; and PCT International Patent Application Nos. PCT/US2015/000320, PCT/US2016/063198, PCT/US2017/036438, PCT/US2010/032320, and PCT/US2012/036007 (all of which are incorporated in their entireties by reference).

### Microbe

In some embodiments, the microbe is any prokaryotic or eukaryotic cell, with any genetic modifications, taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

Generally, although not necessarily, the microbe is a yeast or a bacterium. In some embodiments, the microbe is Rhodosporidium toruloides or Pseudomonas putida. In some embodiments, the microbe is a Gram-negative bacterium. In some embodiments, the microbe is of the phylum Proteobactera. In some embodiments, the microbe is of the class Gammaproteobacteria. In some embodiments, the microbe is of the order Enterobacteriales. In some embodiments, the microbe is of the family Enterobacteriaceae. Examples of suitable bacteria include, without limitation, those species assigned to the Escherichia, Enterobacter, Azotobacter, Erwinia, Bacillus, Pseudomonas, Klebsielia, Proteus, Salmonella, Serratia, Shigella, Rhizobia, Vitreoscilla, and Paracoccus taxonomical classes. Suitable eukaryotic microbes include, but are not limited to, fungal cells. Suitable fungal cells are yeast cells, such as yeast cells of the Saccharomyces genus.

Yeasts suitable for the invention include, but are not limited to, Yarrowia, Candida, Bebaromyces, Saccharomyces, Schizosaccharomyces and Pichia cells. In some embodiments, the yeast is Saccharomyces cerevisae. In some embodiments, the yeast is a species of Candida, including but not limited to C. tropicalis, C. maltosa, C. apicola, C. paratropicalis, C. albicans, C. cloacae, C. guillermondii, C. intermedia, C. lipolytica, C. panapsilosis and C. zeylenoides. In some embodiments, the yeast is Candida tropicalis. In some embodiments, the yeast is a non-oleaginous yeast. In some embodiments, the non-oleaginous yeast is a Saccharomyces species. In some embodiments, the Saccharomyces species is Saccharomyces cerevisiae. In some embodiments, the yeast is an oleaginous yeast. In some embodiments, the oleaginous yeast is a Rhodosporidium species. In some embodiments, the Rhodosporidium species is Rhodosporidium toruloides.

In some embodiments the microbe is a bacterium. Bacterial host cells suitable for the invention include, but are not limited to, Escherichia, Corynebacterium, Pseudomonas, Streptomyces, and Bacillus. In some embodiments, the Escherichia cell is an E. coli, E. albertii, E. fergusonii, E. hermanii, E. marmotae, or E. vulneris. In some embodiments, the Corynebacterium cell is Corynebacterium glutamicum, Corynebacterium kroppenstedtii, Corynebacterium alimapuense, Corynebacterium amycolatum, Corynebacterium diphtheriae, Corynebacterium efficiens, Corynebacterium jeikeium, Corynebacterium macginleyi, Corynebacterium matruchotii, Corynebacterium minutissimum, Corynebacterium renale, Corynebacterium striatum, Corynebacterium ulcerans, Corynebacterium urealyticum, or Corynebacterium uropygiale. In some embodiments, the Pseudomonas cell is a P. putida, P. aeruginosa, P. chlororaphis, P. fluorescens, P. pertucinogena, P. stutzeri, P. syringae, P. cremoricolorata, P. entomophila, P. fulva, P. monteilii, P. mosselii, P. oryzihabitans, P. parafluva, or P. plecoglossicida. In some embodiments, the Streptomyces cell is a S. coelicolor, S. lividans, S. venezuelae, S. ambofaciens, S. avermitilis, S. albus, or S. scabies. In some embodiments, the Bacillus cell is a B. subtilis, B. megaterium, B. licheniformis, B. anthracis, B. amyloliquefaciens, or B. pumilus.

### Biofuel

In some embodiments, the biofuel produced is ethanol, or any other organic molecule, described produced in a cell taught in U.S. Pat. Nos. 7,985,567; 8,420,833; 8,852,902; 9,109,175; 9,200,298; 9,334,514; 9,376,691; 9,382,553; 9,631,210; 9,951,345; and 10,167,488; and PCT International Patent Application Nos. PCT/US14/48293, PCT/US2018/049609, PCT/US2017/036168, PCT/US2018/029668, PCT/US2008/068833, PCT/US2008/068756, PCT/US2008/068831, PCT/US2009/042132, PCT/US2010/033299, PCT/US2011/053787, PCT/US2011/058660, PCT/US2011/059784, PCT/US2011/061900, PCT/US2012/031025, and PCT/US2013/074214 (all of which are incorporated in their entireties by reference).

### Biomass

The biomass can be obtained from one or more feedstock, such as softwood feedstock, hardwood feedstock, grass feedstock, and/or agricultural feedstock, or a mixture thereof.

Softwood feedstocks include, but are not limited to, Araucaria (e.g. A. cunninghamii, A. angustifolia, A. araucana); softwood Cedar (e.g. Juniperus virginiana, Thuja plicata, Thuja occidentalis, Chamaecyparis thyoides Callitropsis nootkatensis); Cypress (e.g. Chamaecyparis, Cupressus Taxodium, Cupressus arizonica, Taxodium distichum, Chamaecyparis obtusa, Chamaecyparis lawsoniana, Cupressus semperviren); Rocky Mountain Douglas fir; European Yew; Fir (e.g. Abies balsamea, Abies alba, Abies procera, Abies amabilis); Hemlock (e.g. Tsuga canadensis, Tsuga mertensiana, Tsuga heterophylla); Kauri; Kaya; Larch (e.g. Larix decidua, Larix kaempferi, Larix laricina, Larix occidentalis); Pine (e.g. Pinus nigra, Pinus banksiana, Pinus contorta, Pinus radiata, Pinus ponderosa, Pinus resinosa, Pinus sylvestris, Pinus strobus, Pinus monticola, Pinus lambertiana, Pinus taeda, Pinus palustris, Pinus rigida, Pinus echinata); Redwood; Rimu; Spruce (e.g. Picea abies, Picea mariana, Picca rubens, Picea sitchensis, Picca glauca); Sugi; and combinations/hybrids thereof.

For example, softwood feedstocks which may be used herein include cedar; fir; pine; spruce; and combinations thereof. The softwood feedstocks for the present invention may be selected from loblolly pine (Pinus taeda), radiata pine, jack pine, spruce (e.g., white, interior, black), Douglas fir, Pinus silvestris, Picea abies, and combinations/hybrids thereof. The softwood feedstocks for the present invention may be selected from pine (e.g. Pinus radiata, Pinus taeda); spruce; and combinations/hybrids thereof.

Hardwood feedstocks include, but are not limited to, Acacia; Afzelia; Synsepalum duloificum; Albizia; Alder (e.g. Alnus glutinosa, Alnus rubra); Applewood; Arbutus; Ash (e.g. F. nigra, F. quadrangulata, F. excelsior, F. pennsylvanica lanceolata, F. latifolia, F. profunda, F. americana); Aspen (e.g. P. grandidentata, P. tremula, P. tremuloides); Australian Red Cedar (Toona ciliata); Ayna (Distemonanthus benthamianus); Balsa (Ochroma pyramidale); Basswood (e.g. T. americana, T. heterophylla); Beech (e.g. F. sylvatica, F. grandifolia); Birch; (e.g. Betula populifolia, B. nigra, B. papyrifera, B. lenta, B. alleghaniensis/B. lutea, B. pendula, B. pubescens); Blackbean; Blackwood; Bocote; Boxelder; Boxwood; Brazilwood; Bubing a; Buckeye (e.g. Aesculus hippocastanum, Aesculus glabra, Aesculus flava/Aesculus octandra); Butternut; Catalpa; Chemy (e.g. Prunus serotina, Prunus pennsylvanica, Prunus avium); Crabwood; Chestnut; Coachwood; Cocobolo; Corkwood; Cottonwood (e.g. Populus balsamifera, Populus deltoides, Populus sargentii, Populus heterophylla); Cucumbertree; Dogwood (e.g. Cornus florida, Cornus nuttallii); Ebony (e.g. Diospyros kurzii, Diospyros melanida, Diospyros crassiflora); Elm (e.g. Ulmus americana, Ulmus procera, Ulmus thomasii, Ulmus rubra, Ulmus glabra); Eucalyptus; Greenheart; Grenadilla; Gum (e.g. Nyssa sylvatica, Eucalyptus globulus, Liquidambar styraciflua, Nyssa aquatica); Hickory (e.g. Carya alba, Carya glabra, Carya ovata, Carya laciniosa); Hornbeam; Hophornbeam; Ipê; Iroko; Ironwood (e.g. Bangkirai, Carpinus caroliniana, Casuarina equisetifolia, Choricbangarpia subargentea, Copaifera spp., Eusideroxylon zwageri, Guajacum officinale, Guajacum sanctum, Hopea odorata, Ipe, Krugiodendronferreum, Lyonothamnus lyonii (L. floribundus), Mesua ferrea, Olea spp., Olneya tesota, Ostrya virginiana, Parrotia persica, Tabebuia serratifolia); Jacarandá; Jotoba; Lacewood; Laurel; Limba; Lignum vitae; Locust (e.g. Robinia pseudacacia, Gleditsia triacanthos); Mahogany; Maple (e.g. Acer saccharum, Acer nigrum, Acer negundo, Acer rubrum, Acer saccharinum, Acer pseudoplatanus); Meranti; Mpingo; Oak (e.g. Quercus macrocarpa, Quercus alba, Quercus stellata, Quercus bicolor, Quercus virginiana, Quercus michauxii, Quercus prinus, Quercus muhlenbergii, Quercus chrysolepis, Quercus lyrata, Quercus robur, Quercus petraea, Quercus rubra, Quercus velutina, Quercus laurifolia, Quercus falcata, Quercus nigra, Quercus phellos, Quercus texana); Obeche; Okoumé; Oregon Myrtle; California Bay Laurel; Pear; Poplar (e.g. P. balsamifera, P. nigra, Hybrid Poplar (Populusxcanadensis)); Ramin; Red cedar; Rosewood; Sal; Sandalwood; Sassafras; Satinwood; Silky Oak; Silver Wattle; Snakewood; Sourwood; Spanish cedar; American sycamore; Teak; Walnut (e.g. Juglans nigra, Juglans regia); Willow (e.g. Salix nigra, Salix alba); Yellow poplar (Liriodendron tulipifera); Bamboo; Palmwood; and combinations/hybrids thereof.

For example, hardwood feedstocks for the present invention may be selected from Acacia, Aspen, Beech, Eucalyptus, Maple, Birch, Gum, Oak, Poplar, and combinations/hybrids thereof. The hardwood feedstocks for the present invention may be selected from Populus spp. (e.g. Populus tremuloides), Eucalyptus spp. (e.g. Eucalyptus globulus), Acacia spp. (e.g. Acacia dealbata), and combinations thereof.

Grass feedstocks include, but are not limited to, C4 or C3 grasses, e.g. Switchgrass, Indiangrass, Big Bluestem, Little Bluestem, Canada Wildrye, Virginia Wildrye, and Goldenrod wildflowers, etc, amongst other species known in the art.

Agricultural feedstocks include, but are not limited to, agricultural byproducts such as husks, stovers, foliage, and the like. Such agricultural byproducts can be derived from crops for human consumption, animal consumption, or other non-consumption purposes. Such crops can be corps such as corn, wheat, sorghum, rice, soybeans, hay, potatoes, cotton, or sugarcane. The feedstock can arise from the harvesting of crops from the following practices: intercropping, mixed intercropping, row cropping, relay cropping, and the like.

In some embodiments, the biomass is an ensiled biomass. In some embodiment, the biomass is ensiled by placing the biomass in an enclosed container or room, such as a silo, or by piling it in a heap covered by an airproof layer, such as a plastic film. The biomass undergoing the ensiling, known as the silage, goes through a bacterial fermentation process resulting in production of volatile fatty acids. In some embodiment, the ensiling comprises adding ensiling agents such as sugars, lactic acid or inculants. In some embodiments, the ensiled biomass comprises one or more toxic compounds. In some embodiments, when ensiled biomass comprises one or more toxic compounds, the microbe is resistant to the one or more toxic compounds.

### REFERENCES CITED HEREIN INCLUDE

[1] C. G. Yoo, X. Meng, Y. Pu, A. J. Ragauskas, Bioresour Technol 2020, 301.
[2] V. Jassal, C. Dou, N. Sun, S. Singh, B. A. Simmons, H. Choudhary, Frontiers in Chemical Engineering 2022, 4.
[3] N. R. Baral, E. R. Sundstrom, L. Das, J. Gladden, A. Eudes, J. C. Mortimer, S. W. Singer, A. Mukhopadhyay, C. D. Scown, ACS Sustain Chem Eng 2019, 7, 9062-9079.
[4] L. Zhao, J. Zhang, D. Zhao, L. Jia, B. Qin, X. Cao, L. Zang, F. Lu, F. Liu, Ind Crops Prod 2022, 188.
[5] H. Choudhary, L. Das, J. G. Pelton, L. Sheps, B. A. Simmons, J. Gladden, S. Singh, Chemistry—A European Journal 2023.
[6] Z. Sun, B. Fridrich, A. de Santi, S. Elangovan, K. Barta, Chem Rev 2018, 118, 614-678.
[7] X. Liu, F. P. Bouxin, J. Fan, V. L. Budarin, C. Hu, J. H. Clark, ChemSusChem 2020, 13, 4296-4317.
[8] T. Renders, G. van den Bossche, T. Vangeel, K. van Aelst, B. Sels, Curr Opin Biotechnol 2019, 56, 193-201.
[9] M. M. Abu-Omar, K. Barta, G. T. Beckham, J. S. Luterbacher, J. Ralph, R. Rinaldi, Y. Román-Leshkov, J. S. M. Samec, B. F. Sels, F. Wang, Energy Environ Sci 2021, 14, 262-292.
[10] H. Luo, E. P. Weeda, M. Alherech, C. W. Anson, S. D. Karlen, Y. Cui, C. E. Foster, S. S. Stahl, J Am Chem Soc 2021, 143, 15462-15470.
[11] X. Du, A. W. Tricker, W. Yang, R. Katahira, W. Liu, T. T. Kwok, P. Gogoi, Y. Deng, ACS Sustain Chem Eng 2021, 9, 7719-7727.
[12] R. Katahira, A. Mittal, K. McKinney, X. Chen, M. P. Tucker, D. K. Johnson, G. T. Beckham, ACS Sustain Chem Eng 2016, 4, 1474-1486.
[13] A. Rodriguez, D. Salvachúa, R. Katahira, B. A. Black, N. S. Cleveland, M. Reed, H. Smith, E. E. K. Baidoo, J. D. Keasling, B. A. Simmons, G. T. Beckham, J. M. Gladden, ACS Sustain Chem Eng 2017, 5, 8171-8180.
[14] H. Suzuki, J. Cao, F. Jin, A. Kishita, H. Enomoto, T. Moriya, J Mater Sci 2006, 41, 1591-1597.
[15] M. S. Kent, J. Zeng, N. Rader, I. C. Avina, C. T. Simoes, C. K. Brenden, M. L. Busse, J. Watt, N. H. Giron, T. M. Alam, M. D. Allendorf, B. A. Simmons, N. S. Bell, K. L. Sale, Green Chemistry 2018, 20, 3024-3037.
[16] D. V. Martinez, A. Rodriguez, M. A. Juarros, E. J. Martinez, T. M. Alam, K. L. Sale, M. S. Kent, A. Rodriguez, B. A. Simmons, K. L. Sale, S. W. Singer, M. S. Kent, B. A. Simmons, S. W. Singer, D. V. Martinez, A. Rodriguez, M. A. Juarros, E. J. Martinez, T. M. Alam, K. L. Sale, M. S. Kent, Green Chemistry 2022, 24, 1627-1643.
[17] J. Zeng, C. G. Yoo, F. Wang, X. Pan, W. Vermerris, Z. Tong, ChemSusChem 2015, 8, 861-871.
[18] R. Ma, M. Guo, X. Zhang, ChemSusChem 2014, 7, 412-415.
[19] M. R. C. Fernandes, X. Huang, H. C. L. Abbenhuis, E. J. M. Hensen, Int J Biol Macromol 2019, 123, 1044-1051.
[20] R. Ma, M. Guo, X. Zhang, Catal Today 2018, 302, 50-60.
[21] M. T. Pope, A. Müller, Angewandte Chemie International Edition in English 1991, 30, 34-48.
[22] S. S. Wang, G. Y. Yang, Chem Rev 2015, 115, 4893-4962.
[23] B. D'Cruz, M. O. Amin, E. Al-Hetlani, Ind Eng Chem Res 2021, 60, 10960-10977.
[24] M. B. Čolović, M. Lacković, J. Lalatović, A. S. Mougharbel, U. Kortz, D. Z. Krstić, Curr Med Chem 2019, 27, 362-379.
[25] Z. Ju, W. Xiao, X. Yao, X. Tan, B. A. Simmons, K. L. Sale, N. Sun, Physical Chemistry Chemical Physics 2020, 22, 2878-2886.
[26] G. F. de Gregorio, R. Prado, C. Vriamont, X. Erdocia, J. Labidi, J. P. Hallett, T. Welton, ACS Sustain Chem Eng 2016, 4, 6031-6036.
[27] D. L. Pavia, G. M. Lampman, G. S. Kriz, J. R. Vyvyan, Introduction to Spectroscopy, 2009.
[28] C. G. Boeriu, D. Bravo, R. J. A. Gosselink, J. E. G. van Dam, Ind Crops Prod 2004, 20, 205-218.
[29] J. C. del Río, J. Rencoret, P. Prinsen, Á. T. Martínez, J. Ralph, A. Gutiérrez, J Agric Food Chem 2012, 60, 5922-5935.
[30] J. L. Wang, L. J. Xu, Crit Rev Environ Sci Technol 2012, 42, 251-325.
[31] A. More, T. Elder, Z. Jiang, Holzforschung 2021, 75, 806-823.
[32] C. H. Calvey, V. Sànchez i Nogué, A. M. White, C. M. Kneucker, S. P. Woodworth, H. M. Alt, C. A. Eckert, C. W. Johnson, Metab Eng 2022.
[33] D. S. Bajwa, G. Pourhashem, A. H. Ullah, S. G. Bajwa, Ind Crops Prod 2019, 139.
[34] M. H. Langholtz, B. J. Stokes, L. M. Eaton, 2016 Billion-Ton Report: Advancing Domestic Resources for a Thriving Bioeconomy, 2016.
[35] P. B. Otoupal, G. M. Geiselman, A. M. Oka, C. A. Barcelos, H. Choudhary, D. Dinh, W. Zhong, H. J. Hwang, J. D. Keasling, A. Mukhopadhyay, E. Sundstrom, R. W. Haushalter, N. Sun, B. A. Simmons, J. M. Gladden, Microb Cell Fact 2022, 21.
[36] H. Choudhary, B. A. Simmons, J. M. Gladden, Front Energy Res 2022, 10, DOI 10.3389/fenrg.2022.868181.
[37] A. Yao, H. Choudhary, M. Mohan, A. Rodriguez, H. Magurudeniya, J. G. Pelton, A. George, B. A. Simmons, J. M. Gladden, ACS Sustain Chem Eng 2021, 9, 4371-4376.
[38] L. E. Walls, P. Otoupal, R. Ledesma-Amaro, S. B. Velasquez-Orta, J. M. Gladden, L. Rios-Solis, Bioresour Technol 2022, 128216.
[39] Solvay Chemicals Inc., HYDROGEN PEROXIDE Concentration Determination 0.1-5% Technical Data Sheet, 2019.
[40] S. Nanayakkara, A. F. Patti, K. Saito, Green Chemistry 2014, 16, 1897-1903.

### Example 1

### An Integrated Chemical and Biological Approach for Biorefinery Lignin Utilization

The efficient utilization of residual lignin is essential for the sustainability of future biorefineries. Several individual biotic and abiotic lignin conversion technologies have been reported. Nevertheless, an integrated approach, as reported in this study, including sequential chemical depolymerization into readily bioavailable molecules followed by their bioconversion is amenable to sustainable biorefinery. First, we explored the potential of polyoxometalate catalyzed oxidative ring opening. Thorough analysis established the feasibility of carboxylic acids production from pure lignin, and subsequently a lignin rich residue from an IL-based biorefinery and raw biomass were investigated. Up to 85 mg carboxylic acids were obtained for every g lignin, while the yields were boosted to about 500 mg and about 1250 mg for biorefinery lignin and raw biomass, respectively. Secondly, the oxidized stream was explored for its bioavailability in the presence of several microorganisms, of which Exophiala alcalophila was the most promising. This study provides an opportunity for the valorization of the 98 Mio. Tons underutilized lignin per year, as currently only 2% of the total lignin produced annually is being utilized.

In this study we employed an integrated approach to depolymerize lignin with a Polyoxometalate Ionic Liquid catalyst ([Ch]₃ [PW₁₂O₄₀]) and subsequently utilize the products for bioconversion.

To establish the reactivity from pure lignin Organosolv lignin (OL) with a reported purity by supplier >99.5% was chosen. The POM-IL is water-insoluble and results in a heterogeneous system facilitating easier analysis and possible non-toxicity for organisms, while no significant difference to the water-soluble acid form could be determined (Tables 6 and 7).

In order to understand the catalytic depolymerization of OL over POM-IL, a mixture of lignin and catalyst (5:2 w/w) was heated at 140° C. for 1 hour in the presence of aqueous H₂O₂ (3.2 wt %).

The residual solids and the oxidized stream (aqueous supernatant) have been separated and analyzed to identify the products of the depolymerization.

The evaluation of the mass of OL after the reaction revealed a conversion of 42%, which is similar with previous oxidative lignin depolymerization studies.^{[20]}

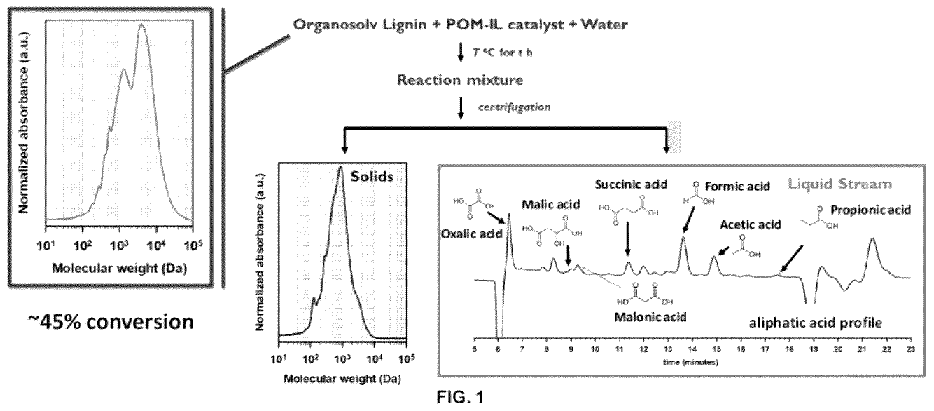

The 2D HSQC NMR profile of the residual solids display a decrease in the signal intensity for the region of S and G units. Additionally, only minor signals for β-O-4 ether are detected while increased C—C structures are present in the residual solids (FIG. 1). The later are formed due to condensation reactions, which are initiated at higher temperatures and the presence of radicals from H₂O₂.^{[6,9]}

The GPC profile of the remaining solids display a breakdown of the higher molecular-weight (MW) lignin (M_{w}=2502 g/mol, PDI=8.96) to lower MW fractions with lower polydispersity (M_{w}=1009 g/mol, PDI=5.33) (FIG. 1). A slight increase in the monomeric region (<200 g/mol) is revealed, but the residual solids contain mostly molecules ranging from 400-1100 g/mol, which can be associated with Di- and trimers but also smaller oligomeric compounds.^{[15,16]}

The IR spectrum of OL detected peaks for the aromatic skeleton at 1600, 1509 and 1116 cm⁻¹ corresponding to the (a-) symmetric aryl ring stretching and C—H bending associated with aromatic skeletons, which are significantly reduced in the residual solids (FIG. 1). Furthermore, an increase in the region of 1700-1715 cm⁻¹ is displayed, which is associated with unconjugated carboxyl groups, suggesting an increase of oxidized structures in the remaining solids.^{[27,28]}

Taking in account the reduced peaks for the aromatic skeleton and the increased oxidized structures as well as the results from GPC and NMR, we have established the depolymerization of pure lignin to non-aromatic, oxidized, lower MW compounds.

To investigate the depolymerized products of the oxidized stream we employed Solution State 2D HSQC NMR and HPLC acid analysis with respective calibration standards and.

The NMR profile of the oxidized stream displays minor signal intensities in the S and G-regions and minor signals in the sidechain region corresponding to —OCH₃ groups, which indicates traces of depolymerized lignin monomers.^{[5,29] Additionally, there are many new signals in the region of δ}_{C}=14-43 ppm/δ_{H}=0.8-2.7 ppm, which can be associated with the C—H bonds of various aliphatic, carboxylic acids.

The acid analysis detected a total aliphatic acid yield of 90 mg acids/g lignin with major products being formic, acetic and oxalic acid at 47,19 and 13 mg/g respectively and minor amounts tartaric, malic, succinic and propionic acid along with aromatic acids. (FIG. 1 and Table 2) It should be highlighted that the oxidized stream may contain complex mixtures of other unidentified smaller and larger organic molecules.

It is generally known that in an acidic environment and the presence of metals H₂O₂ decomposes to active radical species, including OH·, which initiates a chain reaction of various radicals.^{[30,31] Due to the strong electrophilic properties of OH·, it is prone for attacking electron-rich aromatic structures.}

Gregorio et al., which applied a molybdenum POM-IL at 100° C. reported the depolymerization of lignin to aromatic acids and aldehydes, but did not achieve the ring opening reaction, although H₂O₂ was used as oxidant. Other studies employing different catalytic systems and H₂O₂ as an oxidant, depolymerized lignin to mixture of aliphatic acids, aromatic acids, but also phenolic molecules.^{[17,18,20]}

In contrast, our catalytic system resulted in a product profile consisting of carboxylic acids with minimal traces of aromatic acids, concluding that an enhanced ring opening reaction was performed.

In our attempt to valorize the depolymerized products we screened their bioavailability with five organisms, which have been reported for consuming lignin-derived compounds: Exophiala alcalophila, Delftia acidovorans, Pseudonomas putida, Rhodosporidium toruloides and Cupriavidus necator.^{[16,32]}

The main carbon source in the chosen media was the organosolv hydrolysate with a total acid concentration of 370 mg/L. The growth curves and relative biomass gain indicate that under the selected conditions. E. alcalophila and C. necator are promising organisms for consuming the depolymerized acids. However, an acid consumption of over 90% was analyzed for all organisms, while an evaporation of acids is excluded due to the “consumption” of −75% for the control with no cells. (Table 4) These results indicate that the carbon source is available for different organisms, but everyone utilizes the hydrolysate differently.

In 2017 the total supply of available biomass was 365 million dry tons per year of which biorefineries utilize the carbohydrate fraction, while producing lignin-rich streams equaling to 100 million tons/year, but only 2% is used for value added products while the other 98% is unused or burned for generating energy.^{[33,34]} Integrating technologies for valorizing lignin-rich streams is the key for a sustainable and competitive biorefinery. This task is associated with overcoming challenges including the complex nature of the lignin-rich stream and possible inhibitory effects.^{[35,36]} We explored the utilization of a lignin-rich stream of an IL-based biorefinery using Cholinium-Lysinate for pretreatment of sorghum resulting in a lignin-rich residue^{[37,38]}-sorghum lignin-consisting of 5% glucan, 2% xylan and 51% lignin.

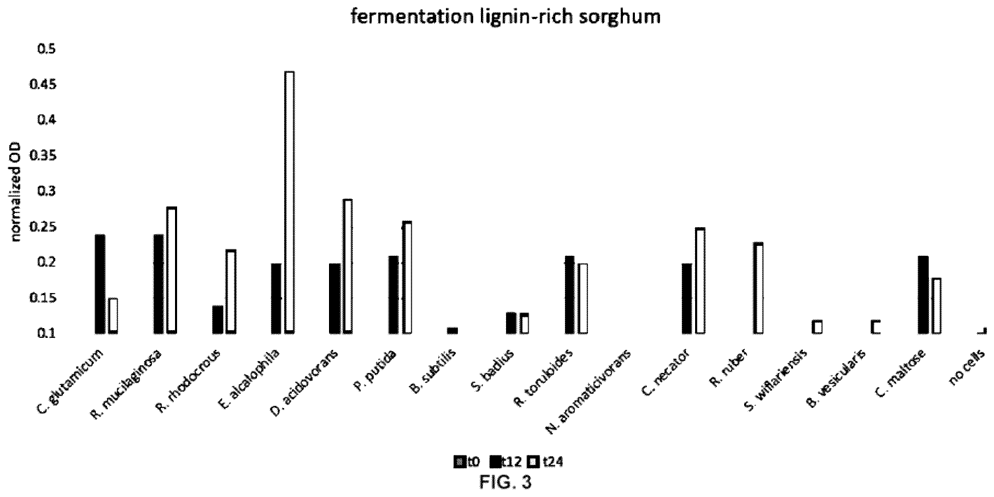

We employed our heterogeneous catalytic system on sorghum lignin at various reaction parameters to identify viable process parameters. (FIG. 3, Tables 8 and 9)^{[39]}

An increase in the conversion of sorghum lignin from 47% to 60% was observed when elevating the oxidant loading by 10-fold from 0.32 to 3.23 wt % H₂O₂. However, a 2.5-fold increase of oxidant from 3.23 to 8.06 wt % resulted in a 33% boost of conversion, while another 2-fold increase only resulted in a minor boost of 5% conversion. This trend can also be observed in the total acids produced, which equal 76, 263, 454 and 475 mg acid/g lignin for 0.32, 3.23, 8,06 and 16.08 wt % H₂O₂, respectively (FIG. 3).

Interestingly the amount of oxalic acid is maximal at 8.06 wt % H₂O₂ while formic acid steadily increases with higher oxidant loadings, which displays further oxidized, low MW products at high oxidant loadings.

The GPC profiles depicts a decreasing trend towards oligomers and monomers for increasing oxidant loadings, but at an oxidant loading of 16.13 wt % a broad peak is displayed (M_{w}=1667 g/mol, PDI=11.1), exceeding the initial sorghum lignin, which indicates that an “overloading” of oxidant results excess of radicals and therefore repolymerization.^{[40]}

Furthermore, were different catalyst loadings and reaction environments explored, but only minor influences on the acid yields were observed. (Tables 8 and 9)

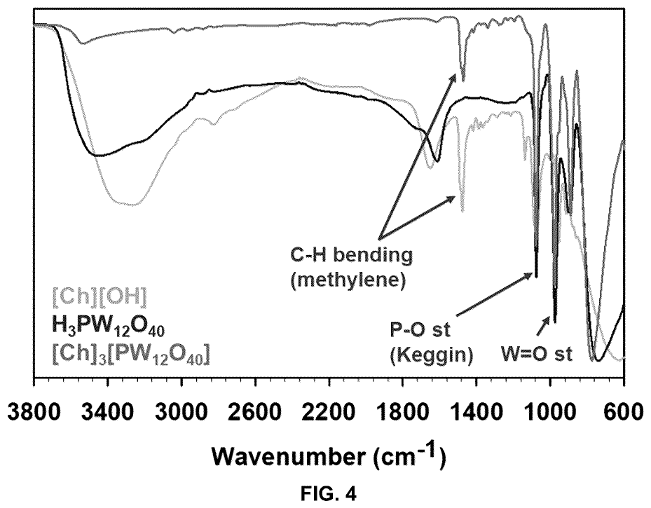

For the bioconversion of oxidized stream from sorghum lignin we extended the tested organisms, but only measured their absolute growth after 12 and 24 hours. (See FIG. 4) In addition to E. alcalophila and C. necator, which already showed promising growth on the oxidized stream of OL, P. putida, R. rhodocorous, R. mucilaginosa and C. glutamicum were able to convert the complex hydrolysate of the lignin-rich stream.

These results indicate an improved conversion of the oxidized stream when a fraction of sugars is present.^{[16]} This study is only the beginning for integrated approaches on lignin conversion, but to valorizing lignin-rich streams is crucial for sustainable biorefineries.

In summary we developed a heterogeneous Polyoxometalate Ionic Liquid catalytic system for depolymerizing lignin to carboxylic acids with a total yield of 454 mg acids per g lignin with 8.06 wt % oxidant loading and biorefinery lignin as substrate. The major acids include oxalic acid, formic acid and acetic acid at 98, 225 and 84 mg/g, respectively, which were subsequently utilized for microbial consumption at over 90% utilization. We proved the catalytic abilities on pure lignin and employed the process on lignin-rich streams from an IL-biorefinery, but we also started the research on applying our Polyoxometalate catalytic system on grassy and woody biomass, to streamline the valorization of biomass for bioconversion.

### Materials

All materials were used as supplied unless otherwise noted. Water was deionized, with specific resistivity of 18 MΩ·cm at 25° C., from PURELAB® Flex (ELGA LabWater, Woodridge, IL). Choline hydroxide (45% in methanol), acetic acid (>99.7%), sodium hydroxide pellets (≥97%), methanol, acetyl bromide (>99%), hydroxylamine (99%), sodium azide, sulfuric acid (98%), organosolv lignin, Phosphotungstic acid hydrate, Formic acid (98.0-100%), DL-Tartaric acid (99%), Malonic acid (99%), Oxalic acid dihydrate (>99.5%), and 30 wt % Hydrogen peroxide solution were obtained from Sigma-Aldrich (St. Louis, MO). Ethanol (200 proof) was purchased from Decon Labs, Inc. (King of Prussia, PA). Sulfuric acid (72%) was procured from RICCA chemical company (Arlington, TX). Amresco, Inc. (Solon, OH) was the source of L-lysine monohydrate. Alkaline lignin was purchased from TCI (Portland, OR). Analytical standard grade glucose and xylose were also obtained from Sigma-Aldrich (St. Louis, MO) and used for calibration.

The biomass (sorghum, poplar, pine, and switchgrass) was dried for 24 h in a 40° C. oven. Subsequently, it was knife-milled with a 2 mm screen (Thomas-Wiley Model 4, Swedesboro, NJ). The resulting biomass was then placed in a leak-proof bag and stored in a dry cool place. Commercial cellulase (Cellic® CTec3) and hemicellulase (Cellic® HTec3) mixtures were provided by Novonesis (Davis, CA).

### Methods

### Synthesis of Catalyst: Polyoxometalate-Ionic Liquid (POM-IL)

The synthesis of Polyoxometalate-Cholinium was conducted as an acid-base reaction of Phosphotungstic acid (H3PW12O40) and Cholinium hydroxide (ChOH). 35 moles Phosphotungstic acid were dissolved in 20 ml dest. H₂O in a 100 ml bottle with a magnetic stir bar, approximately 600 rpm. The bottle was placed in an ice bath to cool the exothermic acid-base reaction. 105 moles ChOH was weight in a glass vial and dissolved with a minimal amount of H₂O to allow the complete transfer of ChOH. Aqueous ChOH was then dispensed slowly into the 100 ml bottle with preloaded, dissolved Phosphotungstic acid. The reaction was completed by additional 2 hours of stirring. The precipitate was separated from the liquid by filtering with a Buchner-funnel using 42-Whatmann filter paper. To ensure the purity of the synthesized POM-IL and remove residual acid the solids were washed with 500 ml H₂O. The solids were dried in a furnace at 40° C. overnight. The structure of the synthesized was confirmed by Fourier-transformation infrared spectroscopy (FIG. 1).

### Generation of Cholinium-Lysinate Pretreated Sorghum Lignin

Sorghum (Sorghum bicolor, donated by Idaho National Labs, Idaho Falls, ID), knife-milled with a 2 mm screen, was used for the generation of cholinium lysinate ([Ch] [Lys])-processed biorefinery lignin. A 1 L Parr 4520 series Bench Top reactor (Parr Instrument Company, model 4871, Moline, IL, USA) was loaded with 15 wt % biomass, 10 wt % [Ch] [Lys] and 75 wt % deionized water and subsequently pretreated for 3 h at 140° C. with stirring at 80 rpm powered by process (Parr Instrument Company, model: 4871) and power controllers (Parr Instrument Company, model: 4875) using three arm, self-centering anchor with Polytetrafluoroethylene (PTFE) wiper blades. After the reaction the reaction volume was cooled down slowly and the pH adjusted to 4.8-5.2. For the enzymatic saccharification and enzyme mix of CTec3: HTec3 (10 mg enzyme per g of biomass, 9:1 v/v, Novozymes North America, Franklinton, NC) was added and heated to 50° C. for 72 hours while mixing at 100 rpm. The reaction volume was transferred to pre-weight centrifugation bottles and centrifuged at 7000 rpm. The supernatant was separated and the lignin rich residue was washed. Therefore the centrifugation bottle was filled with water, mixed thoroughly and centrifuged at 7000 rpm again. The supernatant was discarded and this step repeated until the pH of the supernatant was neutral. The washed solids were frozen at −80° C. overnight. The frozen solids were then dried in a −50° Lipholizer for 3 days. The dried lignin rich residue, termed sorghum lignin, was used as substrate for the depolymerization reactions.

### POM-IL Reactions

All depolymerization reactions were performed in duplicate in a 5000 Multi Reactor (Parr Instrument Company, Moline, Illinois). The reactor was loaded with the according amounts of biomass and catalyst before adding the corresponding hydrogen peroxide/water solution. The reactor was pressured to 20 bar with nitrogen gas and heated to 140° C. for 1 h. During the reaction the reactors were stirred at 480-500 rpm with a magnetic stirrer. After 1 h the reactors were cooled down rapidly with ice to quench the reaction. The reaction volume was transferred in 50 ml centrifugation tubes and centrifuged at 4000 rpm for 10 minutes. The supernatant was separated (Henke-ject 10 ml syringe, nonpyrogenic/nontoxic) and filtered with Thermo Scientific Nalgene™ Syringe Filter (0.45 μm SFCA). The undiluted supernatant was stored for further analysis of the aqueous stream. The solids were washed with deionized water, frozen at −80° C. and lipholized as described above.

### Microbial Cultivations of Oxidized Streams

All used strains for the screening of bioavailability are wildtype strains. They were first cultivated in tubes containing 10 mL of tryptic soy broth from freshly streaked plates and incubated at 30° C. and 200 rpm for 24 hours. The optical densities of a 1:10 dilution was measured to ensure sufficient growth of the cultures. Afterwards the inoculation cultures were prepared by centrifuging 1 ml of the cultivated cultures, separating the media and resuspending the cells in 1 ml dest. H2O. For E. alcalophila the cells were resuspended in 0.5 ml dest. H₂O due to poor growth of the first cultivation. The hydrolysates were adjusted to pH=7, supplemented with YNB-media, which lacked of amino acids, and 50 mM HEPES (each from a 10× stock solution for a 1× final concentration) and diluted with the respective amount of dest. H₂O to achieve a 1:10 dilution of the hydrolysates. The negative control contained the same concentrations of YNB and HEPES, but no hydrolysate. Also a positive control (YNB, HEPES, 500 g/L glucose and 200 g/L fructose) was performed to control the functionality of the cells/cells are growing. Finally, the media was filtered with a 0.2 μm filter to achieve sterility.

For the screening of bioavailability, 780 μL of final media and 20 μL were added to FlowerPlate® 48-well multi well plates (m2p labs, Beckman Coulter Life Sciences, Indianapolis, IN) and covered with sterile AcraSeal™ films (Excel Scientific, Victorville, CA). The plates were incubated for 72 hours in a humidity-controlled incubator with orbital shaking at 900 rpm. The biomass gains and the pH values were measured every 10 minutes. After 72 hours the 48-well plater was put into 4° C. and the media prepared for acid analysis. Therefore the complete volume of each well was transferred, centrifuged and the supernatant diluted 4:1 with the mobile phase (4 mM H₂SO4) for HPLC acid analysis. All cultivations were performed in triplicate.

### Compositional Analysis—Glucan and Xylan

All compositional analysis experiments were conducted in duplicate. Compositional analysis of biomass before and after pretreatment was performed using NREL two-step acid hydrolysis protocols (LAP) LAP-002 and LAP-005 (A. Sluiter, B. Hames, R. Ruiz, C. Scarlata, J. Sluiter, D. Templeton, D. Crocker, Determination of Structural Carbohydrates and Lignin in Biomass: Laboratory Analytical Procedure, 2008). Briefly, 10 mg of biomass and 0.1 mL of 72% sulfuric acid (H₂SO₄) were incubated at 30° C. while shaking at 200 rpm for 1 h. The solution was diluted to 4% H₂SO₄ with 2.5 mL of DI water and autoclaved at 121° C. for 1 h. The reaction was quenched by cooling down the flasks before removing the solids by filtration. The amount of sugars was analyzed on an Agilent HPLC 1260 infinity system (Santa Clara, CA) equipped with a Bio-Rad Aminex HPX-87H column and a Refractive Index detector. An aqueous solution of sulfuric acid (4 mM) was used as the eluent (0.6 mL min-1, column temperature 60° C.). The injection volume was 20 μL with a run time of 20 min. The amount of glucan and xylan was calculated from the glucose and xylose content multiplied by the anhydro correction factors of 162/180 and 132/150, respectively.

### Compositional Analysis—Lignin AcBr

Acetyl bromide-based lignin assay method was employed to determine the lignin content in as reported previously (R. S. Fukushima, M. S. Kerley, M. H. Ramos, J. H. Porter, R. L. Kallenbach, Anim Feed Sci Technol 2015, 201, 25-37. Also cite Alex's ACS sustainable 2021). 10 mg alcohol insoluble biomass residues were weighed in a 2 mL screw cap tubes vial. 1 mL 25% (v/v) acetyl bromide in glacial acetic acid was added to the vials containing biomass samples (Caution: must be operated in fume hood). The vials were sealed and incubated at 50° C. for 2 h with a rotational motion. After 2 h of incubation, vials were cooled in an ice bath for about 5 minutes before centrifuging the samples at 4,000 rpm for 5 minutes. The UV absorbance (at 280 nm) was measured by diluting 6 μL of supernatant with 60 μL master solution (obtained by mixing 48 μL acetic acid, 9.5 μL 2M NaOH and 1.7 μL 0.5M hydroxylamine) and 200 μL glacial acetic acid. It is important to always draw liquids (pipetting) at least 3 times to assure the same amounts of liquid transfer.

The lignin concentration was measured by calibration curve method. In a 2 mL screw cap tubes vial, 10 mg alkaline lignin was treated with 1 mL 25% (v/v) acetyl bromide in glacial acetic acid and incubated at 50° C. for 2 h with a rotational motion. After 2 h of incubation, vials were cooled in an ice bath for about 5 minutes before centrifuging the samples at 14,000 rpm for 5 minutes. Standard samples were prepared by diluting 1, 2, 4, and 6 μL of supernatant with 60 μL master solution and 200 μL glacial acetic acid. UV absorbance was measured at 280 nm and compared against blank (60 μL master solution and 200 μL glacial acetic acid).

### Thermal Gravimetric Analysis (TGA)

Thermal behavior was determined using a Mettler Toledo Stare TGA/DSC1 unit (Mettler Toledo, Leicester, UK) under nitrogen (50 mL/min). Samples between 3 and 10 mg were placed in alumina crucibles (70 μL) and heated from room temperature to 925° C. at a heating rate of 10° C./min. The data was analyzed using STARe Evaluation software.

### Gel Permeation Chromatography (GPC)

GPC was used to determine the relative MW distribution of the sample as described previously (A. Guerra, L. A. Lucia, D. S. Argyropoulos, Holzforschung 2008, 62, 24-30. Also 2023 PdZrP as that was on IL lignin). 10 mg of sample was incubated in 2.5 mL of 92:8 (v/v) acetic acid and acetyl bromide mixture and stirred at 50° C. for 2 h to dissolve lignin. (CAUTION: Acetyl bromide must be operated in fume hood with appropriate PPE. See MSDS for further instructions) Excess of acetyl bromide and acetic acid were removed with a vacuum oven at 40° C., which was connected to a high vacuum pump and a cold trap. This acetylated lignin was dissolved in tetrahydrofuran (THF) and filtered through 0.2 μm polytetrafluoroethylene (PTFE) filters (GE Healthcare Life Sciences, USA). GPC analysis was conducted using a Tosoh Ecosec HLC-8320GPC (Griesheim, Germany) equipped with Agilent PLgel 5 μm Mixed-D and a Diode array detector. THF (spiked with 250 ppm of butylated hydroxytoluene) was used as the eluent (1 mL min-1, column temperature 40° C.). The GPC standards, which contained polystyrene ranging from 162 to 69,650 g mol-1, were purchased from Agilent and used for calibration.

### High Pressure Liquid Chromatography

The sugar and acid analysis were conducted on a Agilent 1260 Infinity II Prime LC system equipped with a RID and DAD detector and Aminex HPX-87H column heated to 60° C. Calibration series for glucose, xylose, oxalic acid, tartaric acid, malic acid, succinic acid, formic acid, acetic acid and propionic acid were prepared. The samples of the POM-IL reactions have been diluted 10-fold in the mobile phase (4 mM sulfuric acid) and then filtered in centrifugal filter tubes (0.45 μm, obtained from VWR North America) before injecting the sample.

### Infrared Spectroscopy

The identity of the synthesized catalyst was established using FT-IR spectroscopy using a Bruker VERTEX 70/80 system (Billerica, MA). The data was analyzed using OPUS (version 8.2, build 8, 2, 28 (20190310) software. Additionally, FT-IR spectra of a few selected samples were recorded.

### 2D HSQC NMR

The ground material was dispersed in DMSO-d6 and allowed to stand overnight to extract lignin. For the lignin oil after hydrogenolysis, the dried oil was directly dissolved in DMSO-d6 for the NMR analysis. The 2D heteronuclear single quantum coherence (HSQC) spectra were collected on a Bruker Avance I 800 MHz spectrometer equipped with a TXI probe at 310K. A standard Bruker pulse sequence (hsqcetgpsisp2.2) was used with the following parameters which are typical for plant cell wall samples. HSQC spectra were collected from 11 to −1 ppm in F₂ (¹H) dimension with 1024 data points for 53 ms acquisition time, and from 165 to −10 ppm in F₁ (¹³C) dimension with 256 data points for 3.5 ms acquisition time. A total of 256 scans were recorded for each t1 point with a pulse delay of 1 s. The central DMSO solvent peak was used as a reference for the chemical shift calibration for all samples (δ_{C} 39.5 ppm, δ_{H} 2.5 ppm). All HSQC spectra were processed using typical 90° sine square apodization in both F2 and F1 dimensions and the contours were integrated in the MestreNOVA software (v.14). Peaks were assigned according to published data.

Determination of H₂O₂ Concentration

The experimental instructions of Solvay Chemicals, Inc. for the determination of hydrogen peroxide concentration (0.1-5%) were used to determine the concentration of the remaining hydrogen peroxide after the reaction in the oxidized stream (Solvay Chemicals Inc., HYDROGEN PEROXIDE Concentration Determination 0.1-5% Technical Data Sheet, 2019).

### Calculations

The following equations were used for calculation the conversions:

$$recovered solids = \frac{[ empty vial ] - [ vial after drying ]}{0.95}$$

$$unreacted solids = recovered solids - 0.9 * [ catalyst ]$$

$$conversion [ \% ] = ( 1 - \frac{unreacted solids}{[ biomass ]} ) * 100$$

The removal of components of the compositional analysis was calculated with the following equation:

$$removal [ \% ] = ( 1 - \frac{{X}_{after reaction}}{{X}_{before reaction}} ) * 100$$

$$X = glucan [ \% ] / xylan [ \% ] / lignin [ \% ] .$$

### Experimental Results

TABLE 1
Detected aliphatic carboxylic acid from HPLC acid analysis.

| Chemical Name | Chemical Structure | Retention time [s] |
|---|---|---|
| Oxalic Acid | 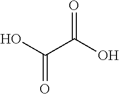 | 6.202 |
| Tartaric Acid | 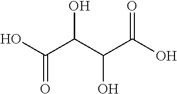 | 8.111 |
| Malic Acid | 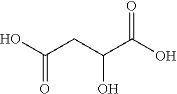 | 9.207 |
| Succinic Acid | 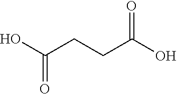 | 11.365 |
| Formic Acid | 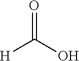 | 13.525 |
| Acetic Acid | 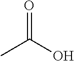 | 14.874 |
| Propionic Acid | 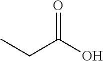 | 17.479 |

TABLE 2
Analysis of residual solids and oxidized stream from
reaction for proof of concept. Reactions were performed
at 140° C., 1h, 20 bar N₂, with 5 wt % organosolv lignin,
3.23 wt % H₂O₂ and 2 wt % POM-IL.

| Conversion [%] | | | 41.83 ± 0.42 |
|---|---|---|---|
| | Acid profile [mg/g] | Oxalic Acid | 11.91 ± 0.29 |
| | | Tartaric Acid | 5.14 ± 0.26 |
| | | Malic Acid | 3.94 ± 0.11 |
| | | Succinic Acid | 0.88 ± 0.01 |
| | | Formic Acid | 50.87 ± 2.04 |
| | | Acetic Acid | 21.49 ± 0.05 |
| | | Propionic Acid | 0.86 ± 0.09 |
| | Molecular Weight | M_{W} [g/mol] | 1009 |
| | Distribution | M_{N} [g/mol] | 189 |
| | | PDI | 5.33 |

TABLE 3
Maximal biomass gain from fermentations for proof of concept.

| Organism | Maximal biomass gain [rel. OD₅₉₅] |
|---|---|
| E. alcalophila | 0.71 ± 0.03 |
| D. acidovorans | 0.29 ± 0.03 |
| P. putida | 0.37 ± 0.01 |
| R. toruloides | 0.29 ± 0.02 |
| C. necator | 0.61 ± 0.13 |

TABLE 4
Acid analysis from fermentation for proof of concept.

| | | Total acids [mg/L] | Consumed acids [%] |
|---|---|---|---|
| T = 0 | | 372.78 | |
| After 72 h | E. alcalophila | 22.62 ± 7.20 | 93.93 ± 1.93 |
| fermentation | D. acidovorans | 22.11 ± 9.64 | 94.07 ± 2.59 |
| | P. putida | 26.38 ± 2.21 | 92.92 ± 0.59 |
| | R. toruloides | 29.74 ± 6.13 | 92.02 ± 1.64 |
| | C. necator | 28.14 ± 4.44 | 92.45 ± 1.19 |
| | No cells | 650.83 ± 47.60 | −74.59 ± 12.77 |

TABLE 5
Compositional analysis of sorghum lignin.

| Glucan [%] | Xylan [%] | Lignin [%] | Ash [%] |
|---|---|---|---|
| 4.74 ± 0.17 | 1.87 ± 0.01 | 51.59 ± 2.02 | 15.75 ± 0.45 |

Table 6 shows the analysis of residual solids from control reactions. Reactions were performed at 140° C., 1 h, 20 bar N₂ with 5 wt % sorghum lignin. The catalyst was the synthesized POM-IL. ^{a}used catalyst was the Polyoxometalate in its acid form; ^{b}reaction environment was 2 bar O₂.

Table 7 shows the analysis of oxidized stream from control reactions. Reactions were performed at 140° C., 1 h, 20 bar N₂ with 5 wt % sorghum lignin. The catalyst was the synthesized POM-IL. ^{a}used catalyst was the Polyoxometalate in its acid form; ^{b}reaction environment was 2 bar O₂.

Table 8 shows the analysis of residual solids from sorghum lignin reactions. Reactions were performed at 140° C., 1 h, 20 bar N₂ with 5% sorghum lignin. The catalyst was the synthesized POM-IL

Table 9 shows the analysis of oxidized stream from sorghum lignin reactions. Reactions were performed at 140° C., 1 h, 20 bar N₂ with 5% sorghum lignin. The catalyst was the synthesized POM-IL.

TABLE 10
Analysis of raw biomass.

| | Composition | | |
|---|---|---|---|
| | Glucan [%] | Xylan [%] | Lignin [%] |
| Switchgrass | 21.59 ± 1.00 | 11.82 ± 0.09 | 20.11 ± 2.70 |
| Sorghum | 31.67 ± 1.54 | 18.75 ± 1.06 | 25.88 ± 0.72 |
| Poplar | 36.82 ± 1.18 | 13.25 ± 0.75 | 18.85 ± 0.35 |
| Pine | 35.49 ± 3.31 | 16.37 ± 0.87 | 40.02 ± 1.07 |

Table 11 shows the analysis of residual solids from raw biomass reactions reactions were performed at 140° C., 1 h, 20 bar N₂ with 3.23 wt % H₂O₂ and 2 wt % POM-IL.

Table 12 shows the analysis of oxidized stream from raw biomass reactions reactions were performed at 140° C. 1 h. 20 bar N₂ with 3.23 wt % H₂O₂ and 2 wt % POM-IL.

TABLE 6
Analysis of residual solids from control reactions.

| | catalyst | H₂O₂ | | | | | | | |
|---|---|---|---|---|---|---|---|---|---|
| | loading | loading | Conversion | Composition | | | Removal [%] | | |
| 6Entry | [wt %] | [wt %] | [%] | Glucan [%] | Xylan [%] | Lignin [%] | Glucan [%] | Xylan [%] | Lignin [%] |
| 1 | 0 | 0 | 19.34 ± 0.02 | 6.70 ± 1.86 | 1.93 ± 0.11 | 17.70 ± 0.07 | 81.13 ± 5.25 | 88.19 ± 0.67 | 55.78 ± 0.18 |
| 2 | 2^{a} | 3.23 | 56.77 ± 1.67 | 6.06 ± 0.15 | 0.91 ± 0.05 | 15.26 ± 0.83 | 82.91 ± 0.43 | 94.43 ± 0.31 | 61.85 ± 2.07 |
| 3^{b} | 2 | 3.23 | 57.77 ± 0.05 | 3.67 ± 0.00 | 1.60 ± 0.00 | 15.66 ± 0.69 | 89.66 ± 0.01 | 90.23 ± 0.01 | 60.86 ± 1.73 |

TABLE 7
Analysis of oxidized stream from control reactions.

| | catalyst | H₂O₂ | | Acid profile [mg/g lignin] | | | | | | |
|---|---|---|---|---|---|---|---|---|---|---|
| | loading | loading | Conversion | Oxalic | Tartaric | Malic | Succinic | Formic | Acetic | Propionic |
| Entry | [wt %] | [wt %] | [%] | Acid | Acid | Acid | Acid | Acid | Acid | Acid |
| 1 | 0 | 0 | 19.34 ± 0.02 | 0.68 ± 0.47 | 0.22 ± 0.32 | 3.24 ± 0.54 | 0.03 ± 0.00 | 8.23 ± 0.79 | 5.84 ± 0.66 | 0.00 ± 0.00 |
| 2 | 2^{a} | 3.23 | 56.77 ± 1.67 | 68.60 ± 5.71 | 5.49 ± 0.05 | 14.78 ± 0.46 | 0.83 ± 0.03 | 107.1 ± 1.54 | 31.21 ± 0.59 | 4.31 ± 0.21 |
| 3^{b} | 2 | 3.23 | 57.77 ± 0.05 | 0.00 ± 0.00 | 5.01 ± 0.15 | 16.06 ± 2.61 | 0.68 ± 0.04 | 96.93 ± 0.45 | 30.78 ± 0.85 | 5.18 ± 0.70 |

TABLE 8
Analysis of residual solids from sorghum lignin reactions.

| | catalyst | H₂O₂ | | Composition | | |
|---|---|---|---|---|---|---|
| | loading | loading | Conversion | Glucan | Xylan | Lignin |
| Entry | [wt %] | [wt %] | [%] | [%] | [%] | [%] |
| 1 | 2 | 0 | 41.53 ± 0.29 | 3.91 ± 0.17 | 0.80 ± 1.14 | 18.11 ± 4.04 |
| 2 | | 0.32 | 41.06 ± 0.84 | 3.82 ± 0.11 | 0.79 ± 1.12 | 14.95 ± 3.38 |
| 3 | | 1.61 | 46.75 ± 0.20 | 3.84 ± 0.12 | 1.59 ± 0.03 | 6.46 ± 1.51 |
| 4 | | 3.23 | 60.01 ± 2.32 | 3.91 ± 0.14 | 1.61 ± 0.04 | 4.23 ± 1.77 |
| 5 | | 8.06 | 93.27 ± 0.11 | 3.24 ± 0.18 | 0.59 ± 0.00 | 7.72 ± 0.96 |
| 6 | | 16.13 | >95 | 4.66 ± 0.14 | 0.61 ± 0.00 | 7.57 ± 0.23 |
| 7 | 0 | 3.23 | 46.02 ± 0.04 | 4.02 ± 0.03 | 0.79 ± 1.12 | 26.12 ± 2.95 |
| 8 | 1 | | 53.02 ± 0.00 | 4.24 ± 0.98 | 0.76 ± 0.09 | 6.51 ± 0.86 |
| 9 | 5 | | 68.79 ± 0.78 | 4.36 ± 0.06 | 0.73 ± 0.05 | 20.55 ± 3.95 |
| 10 | 10 | | 48.29 ± 0.16 | 2.87 ± 0.16 | 0.64 ± 0.03 | 5.44 ± 3.46 |

| | Removal [%] | | | Molecular Weight | | |
|---|---|---|---|---|---|---|
| | Glucan | Xylan | Lignin | Distribution | | |
| Entry | [%] | [%] | [%] | M_{W} | M_{N} | PDI |
| 1 | 17.64 ± 3.61 | 56.98 ± 60.84 | 64.89 ± 7.83 | 884 | 200 | 4.42 |
| 2 | 19.51 ± 2.40 | 57.65 ± 59.90 | 71.02 ± 6.55 | 939 | 190 | 4.94 |
| 3 | 19.05 ± 2.60 | 14.63 ± 1.64 | 87.49 ± 2.93 | 800 | 139 | 5.75 |
| 4 | 17.58 ± 3.03 | 13.85 ± 2.08 | 91.80 ± 3.43 | 866 | 138 | 6.29 |
| 5 | 31.69 ± 3.73 | 68.27 ± 0.24 | 85.03 ± 1.87 | 1222 | 142 | 8.58 |
| 6 | 1.72 ± 2.93 | 67.52 ± 0.01 | 85.33 ± 0.44 | 1667 | 150 | 11.13 |
| 7 | 15.19 ± 0.57 | 57.59 ± 59.98 | 49.38 ± 5.72 | | | |
| 8 | 10.58 ± 20.62 | 59.11 ± 4.70 | 87.38 ± 1.67 | | | |
| 9 | 8.10 ± 1.29 | 60.84 ± 2.51 | 60.17 ± 7.66 | | | |
| 10 | 39.53 ± 3.34 | 65.87 ± 1.68 | 89.46 ± 6.70 | | | |

TABLE 9
Analysis of oxidized stream from sorghum lignin reactions.

| | catalyst | H₂O₂ | | Acid profile [mg/g lignin] | | |
|---|---|---|---|---|---|---|
| | loading | loading | Conversion | Oxalic | Tartaric | Malic |
| Entry | [wt %] | [wt %] | [%] | Acid | Acid | Acid |
| 1 | 2 | 0 | 41.53 ± 0.29 | 12.92 ± 0.40 | 0.00 ± 0.00 | 1.93 ± 0.13 |
| 2 | | 0.32 | 41.06 ± 0.84 | 15.82 ± 0.46 | 0.00 ± 0.00 | 3.06 ± 0.14 |
| 3 | | 1.61 | 46.75 ± 0.20 | 35.89 ± 1.24 | 2.24 ± 0.05 | 10.25 ± 0.79 |
| 4 | | 3.23 | 60.01 ± 2.32 | 76.92 ± 7.39 | 7.57 ± 0.82 | 12.80 ± 1.01 |
| 5 | | 8.06 | 93.27 ± 0.11 | 97.69 ± 2.71 | 13.61 ± 2.12 | 10.76 ± 0.41 |
| 6 | | 16.13 | 102.85 ± 0.86 | 76.40 ± 0.11 | 9.09 ± 3.59 | 11.28 ± 2.06 |
| 7 | 0 | 3.23 | 46.02 ± 0.04 | 33.08 ± 0.19 | 2.74 ± 0.42 | 18.26 ± 0.50 |
| 8 | 1 | | 53.02 ± 0.00 | 65.57 ± 0.90 | 6.08 ± 0.34 | 12.21 ± 2.83 |
| 9 | 5 | | 68.79 ± 0.78 | 86.70 ± 2.50 | 8.47 ± 0.10 | 15.64 ± 0.94 |
| 10 | 10 | | 48.29 ± 0.16 | 88.36 ± 6.29 | 8.38 ± 0.30 | 15.60 ± 2.74 |

| | Acid profile [mg/g lignin] | | | |
|---|---|---|---|---|
| | Succinic | Formic | Acetic | Propionic |
| Entry | Acid | Acid | Acid | Acid |
| 1 | 0.03 ± 0.00 | 8.09 ± 0.47 | 4.71 ± 0.08 | 0.82 ± 0.99 |
| 2 | 0.07 ± 0.01 | 12.14 ± 0.22 | 5.17 ± 0.40 | 0.62 ± 0.07 |
| 3 | 0.34 ± 0.01 | 44.77 ± 2.72 | 12.64 ± 0.12 | 3.02 ± 0.44 |
| 4 | 0.89 ± 0.06 | 104.84 ± 11.17 | 31.71 ± 3.67 | 5.56 ± 0.76 |
| 5 | 3.01 ± 0.07 | 224.98 ± 1.45 | 84.25 ± 0.94 | 9.07 ± 0.32 |
| 6 | 3.78 ± 0.14 | 250.51 ± 1.91 | 98.31 ± 2.65 | 13.16 ± 0.76 |
| 7 | 0.98 ± 0.01 | 113.98 ± 0.77 | 32.61 ± 1.60 | 6.16 ± 0.83 |
| 8 | 0.89 ± 0.01 | 102.97 ± 0.87 | 31.43 ± 0.01 | 5.44 ± 0.16 |
| 9 | 0.94 ± 0.12 | 111.91 ± 1.87 | 34.39 ± 2.65 | 5.38 ± 0.33 |
| 10 | 0.90 ± 0.17 | 113.89 ± 2.77 | 35.16 ± 1.03 | 6.66 ± 0.18 |

TABLE 11
Analysis of residual solids from raw biomass reactions.

| | | Conversion | Composition | | | Removal [%] | | |
|---|---|---|---|---|---|---|---|---|
| Entry | substrate | [%] | Glucan [%] | Xylan [%] | Lignin [%] | Glucan [%] | Xylan [%] | Lignin [%] |
| 1 | Switchgrass | 75.45 ± 0.51 | 45.19 ± 0.03 | 1.56 ± 0.07 | 17.92 ± 0.87 | −109.33 ± 0.12 | 86.84 ± 0.56 | 10.89 ± 4.31 |
| 2 | Sorghum | 83.10 ± 3.25 | 49.22 ± 0.03 | 2.32 ± 0.28 | 26.21 ± 8.13 | −55.40 ± 0.08 | 87.63 ± 1.51 | −1.26 ± 31.41 |
| 3 | Poplar | 66.80 ± 0.14 | 51.85 ± 1.40 | 1.25 ± 0.00 | 22.95 ± 1.31 | −40.82 ± 3.82 | 90.53 ± 0.02 | −21.71 ± 6.93 |
| 4 | Pine | 67.99 ± 0.19 | 50.88 ± 0.13 | 1.59 ± 0.03 | 33.32 ± 10.03 | −43.36 ± 0.36 | 90.31 ± 0.17 | 16.72 ± 25.08 |

TABLE 12
Analysis of oxidized stream from raw biomass reactions.

| | | | | Acid profile [mg/g] | | | | | | |
|---|---|---|---|---|---|---|---|---|---|---|
| | | Conversion | | Oxalic | Tartaric | Malic | Succinic | Formic | Acetic | Propionic |
| Entry | substrate | [%] | | Acid | Acid | Acid | Acid | Acid | Acid | Acid |
| 1 | Switchgrass | 75.45 | ±0.51 | 112.55 ± 4.59 | 61.06 ± 0.84 | 260.91 ± 22.14 | 1.31 ± 0.00 | 493.26 ± 3.26 | 217.45 ± 2.34 | 2.84 ± 1.46 |
| 2 | Sorghum | 83.10 | ±3.25 | 116.15 ± 5.76 | 48.85 ± 2.51 | 156.41 ± 11.53 | 1.26 ± 0.11 | 347.62 ± 5.47 | 119.15 ± 4.20 | 1.86 ± 0.55 |
| 3 | Poplar | 66.80 | ±0.14 | 80.58 ± 1.26 | 56.33 ± 0.57 | 208.60 ± 5.79 | 1.87 ± 0.04 | 557.05 ± 2.22 | 348.47 ± 1.02 | 0.00 ± 0.00 |
| 4 | Pine | 67.99 | ±0.19 | 38.98 ± 2.66 | 20.41 ± 0.07 | 155.46 ± 4.67 | 0.73 ± 0.03 | 259.36 ± 5.88 | 97.83 ± 1.77 | 0.00 ± 0.00 |

It is to be understood that, while the invention has been described in conjunction with the preferred specific embodiments thereof, the foregoing description is intended to illustrate and not limit the scope of the invention. Other aspects, advantages, and modifications within the scope of the invention will be apparent to those skilled in the art to which the invention pertains.

All patents, patent applications, and publications mentioned herein are hereby incorporated by reference in their entireties.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method to deconstruct a biomass: the method comprising: introducing a solvent comprising a polyoxometalate (POM) ionic liquid (POM-IL) to a biomass to produce a mixture, such that the POM-IL oxidizes the biomass to produce a carboxylic acid.

2. The method of claim 1, wherein the biomass is a lignocellulosic biomass.

3. The method of claim 1, wherein the carboxylic acid is an aliphatic carboxylic acid or aromatic carboxylic acid, or a mixture thereof.

4. The method of claim 1, wherein the carboxylic acid has 1, 2, 3, 4, 5, 6, 7, 8, 9, or 10 carbon atoms, or a mixture thereof.

5. The method of claim 3, wherein the aliphatic carboxylic acid is a monocarboxylic acid.

6. The method of claim 5, wherein the monocarboxylic acid is a formic, acetic, malic, or propionic acid, or a mixture thereof.

7. The method of claim 3, wherein the aliphatic carboxylic acid is a dicarboxylic acid.

8. The method of claim 7, wherein the dicarboxylic acid is a oxalic, malonic, succinic, malic, or tartaric acid, or a mixture thereof.

9. The method of claim 3, wherein the aromatic acid is a hydroxybenzoic acid, vanillic acid, p-coumaric acid, ferulic acid, syringic acid, or a mixture thereof.

10. The method of claim 1, wherein the method results in the mixture comprising a carboxylic acid, or mixture thereof, and unreacted or not oxidized biomass.

11. The method of claim 2, wherein the lignocellulosic biomass comprises untreated and/or raw agricultural and forest residue, or residues obtained from a biorefinery or paper and pulp.

12. The method of claim 1, wherein the method deconstructs untreated and/or raw biomass to a carboxylic acid in aqueous and glucan rich solid residue.

13. The method of claim 1, wherein the method further comprises: separating the carboxylic acid from the mixture.

14. The method of claim 1, wherein the carboxylic acid is biocompatible for further conversion.

15. A composition comprising: (a) a solvent comprising a polyoxometalate (POM) ionic liquid (POM-IL), and (b) a biomass.

16. The composition of claim 15, further comprising a carboxylic acid, wherein the carboxylic acid is produced from the POM-IL oxidizing the biomass.
